(19) United States
(12) Patent Application Publication
(10) Pub. No.: US 2026/0273253 A1
(43) Pub. Date: Sep. 17, 2026

(54) DISSOLVABLE MICRONEEDLE PATCH FOR LARGE MOLECULE THERAPEUTIC DELIVERY

(71) Applicant: Maman Biomedical Inc., Toronto (CA)

(72) Inventor: Latchmi Raghunanan, Toronto (CA)

(73) Assignee: Maman Biomedical Inc., Toronto, ON (CA)

(21) Appl. No.: 19/553,637

(22) Filed: Mar. 2, 2026

Related U.S. Application Data
(60) Provisional application No. 63/764,658, filed on Feb. 28, 2025.

(51) Int. Cl. A61M 37/00; A61K 9/00; A61K 31/568; A61K 31/57; A61K 38/24; A61K 47/38; A61P 5/02; A61P 5/06; A61P 15/08; A61P 15/12

(52) U.S. Cl. CPC A61M 37/0015; A61K 9/0021; A61K 31/568; A61K 31/57; A61K 38/24; A61K 47/38; A61P 5/02; A61P 5/06; A61P 15/08; A61P 15/12; A61M 2037/0023; A61M 2037/0046; A61M 2037/0053; A61M 2037/0061

(57) ABSTRACT

The present disclosure provides a dissolvable microneedle patch for intradermal administration of at least one large molecule therapeutic to a subject. The dissolvable microneedle patch includes a substantially planar base layer and a plurality of dissolvable microneedles extending from the base layer. The microneedles include a matrix material including high molecular weight, non-crosslinked hyaluronic acid and methylcellulose. The microneedles include at least one large molecule therapeutic optionally encapsulated within a biodegradable polymer. The plurality of microneedles are configured to penetrate skin layers and dissolve following administration to release the therapeutic within intradermal tissue, enabling sustained therapeutic delivery without chemical modification of the therapeutic agent or retained implantable structures. The patch optionally includes an adhesive surface and at least one sensor configured to detect biological markers or hormone concentrations from bodily fluids.

### CROSS-REFERENCE TO RELATED APPLICATIONS

The present application claims the benefit of U.S. Provisional Patent Application No. 63,764,658, titled “DISSOLVABLE MICRONEEDLE PATCH FOR LARGE MOLECULE THERAPEUTIC DELIVERY” and filed on Feb. 28, 2025, all of which are hereby incorporated by reference in their entirety for all purposes.

### TECHNICAL FIELD

The present invention relates to devices for drug delivery and methods and uses thereof. More specifically, the present invention relates to dissolvable microneedle patches for delivery of large molecule therapeutics including hormones.

### BACKGROUND

Drug delivery continues to evolve toward minimally invasive systems. Patients increasingly prefer needle-free or reduced-needle technologies. Conventional transdermal patches rely on passive diffusion across the stratum corneum. The stratum corneum blocks most hydrophilic and large-molecule therapeutics. So, passive patches deliver only small, lipophilic, low-dose drugs with reliability. The large-molecule therapeutics include proteins, peptides, hormones, enzymes, and biologics. The large-molecule therapeutics must reach viable tissue layers to achieve systemic exposure. However, passive diffusion systems cannot transport intact macromolecules across the skin barrier. Clinical practice relies heavily on subcutaneous or intramuscular injection for large-molecule drugs. Repeated injections increase patient discomfort and reduce compliance.

In recent times, microneedle patch systems have gained significant attention. A microneedle patch carries an array of microneedles arranged on a supporting substrate. The microneedle array creates micro-scale pathways across the stratum corneum without hypodermic needles. These microneedles are dissolvable in nature. The dissolving microneedle patches integrate therapeutic agents into polymer matrices that dissolve after insertion. Dissolving formats eliminate sharps waste and avoid retained hardware.

However, the dissolving microneedle patches must balance mechanical strength and solubility within each microneedle in the array. The microneedles must penetrate skin without bending or fracturing. Also, the same microneedles must dissolve after insertion to release drug payload. Increasing polymer concentration improves strength but slows dissolution. Further, reducing polymer density accelerates dissolution but weakens insertion. Hydrophilic matrices preserve large-molecule stability yet reduce structural robustness.

Furthermore, high-dose large-molecule therapies constrain microneedle patch design. Many hormonal and biologic treatments require milligram-scale loading or repeated dosing. Also, limited microneedle volume restricts total drug capacity per patch. Increased loading disrupts polymer continuity and weakens microneedle integrity. High protein concentration accelerates water uptake and premature softening. Further, enlarging the microneedle size or density to increase loading introduces fabrication defects and insertion variability across the patch.

Non-uniform insertion within a microneedle patch results in uneven intradermal deposition. Partial insertion or premature dissolution compromises dose accuracy. Some sustained-release microneedle patch systems rely on retained hardware or detachable structures. The retained components increase regulatory complexity and complicate repeat administration. Furthermore, manufacturing variability affects the microneedle patch integrity. Changes in casting, drying, or mold filling alter array uniformity and mechanical performance, especially under high large-molecule loading conditions.

In light of the above stated discussion, there exists a need for overcoming the above stated drawbacks of the current microneedle patches.

### SUMMARY

In an aspect, a dissolvable microneedle patch for intradermal administration of a hormone to a subject is disclosed. The microneedle patch includes a substantially planar base layer and a plurality of microneedles extending from a surface of the base layer. Each of the plurality of microneedles has a height of 1000-3000 μm100. Each of the plurality of microneedles includes a solidified matrix. The solidified matrix includes 0.5-20% w/w non-crosslinked high molecular weight hyaluronic acid. The non-crosslinked high molecular weight hyaluronic acid has a molecular weight of 1000-1400 kDa. In addition, the matrix includes 0.5-20% w/w methylcellulose. The methylcellulose has a viscosity of 100-8000 cP. Further, the matrix includes 0-20% w/w non-crosslinked low molecular weight hyaluronic acid. The non-crosslinked low molecular weight hyaluronic acid has a molecular weight of 20-1000 kDa. The matrix includes the hormone and no added chemical crosslinking agents. Less than 100% of the hormone is encapsulated in at least one biodegradable polymer dispersed within the matrix. A non-encapsulated portion of the hormone is present within the matrix material, and an encapsulated portion of the hormone is contained within the biodegradable polymer. The plurality of microneedles dissolve within 1-14 days following insertion into intradermal tissue and the biodegradable polymer degrades within 1-14 days following insertion.

In an embodiment of the present disclosure, the plurality of microneedles have a pitch in a range of 100-3000 μm and a base width of 100-500 μm.

In an embodiment of the present disclosure, the microneedle patch includes 50-1000 microneedles.

In an embodiment of the present disclosure, the microneedles maintain structural integrity during insertion into intradermal tissue.

In an embodiment of the present disclosure, the base layer and the plurality of microneedles include the matrix and the plurality of microneedles are congruous extensions of the base layer.

In an embodiment of the present disclosure, the base layer includes an adhesive surface configured to secure the microneedle patch to skin. The adhesive surface forms a perimeter or partial perimeter around the base layer.

In an embodiment of the present disclosure, the microneedle patch further includes a non-adhesive top layer.

In an embodiment of the present disclosure, each of the plurality of microneedles include a tapered tip having a contact area less than that of a 30-gauge hypodermic needle.

In an embodiment of the present disclosure, each of the plurality of microneedles include a tip portion that constitutes 5-95% of the total volume of the plurality of microneedles.

In an embodiment of the present disclosure, the plurality of microneedles are boreless.

In an embodiment of the present disclosure, the base layer has a surface area of approximately 4 cm×4 cm, 3 cm×3 cm, or 1.5 cm×1.5 cm.

In an embodiment of the present disclosure, each of the plurality of microneedles contain an equal or approximately equal amount of the hormone.

In an embodiment of the present disclosure, the plurality of microneedles have a length configured to terminate in a dermis following insertion into the skin.

In an embodiment of the present disclosure, the base layer further includes at least one sensor configured to detect at least one of hormone concentration in blood, interstitial fluid, or sweat, a biological marker associated with endocrine function, and a temperature of the interstitial fluid.

In an embodiment of the present disclosure, the biodegradable polymer includes at least one of polyethylene glycol (PEG), polyvinyl alcohol (PVA), poly(lactic-co-glycolic acid) (PLGA), polycaprolactone (PCL), poly(lactic acid) (PLA), polyanhydride, PMMA, PHEMA, polyurethanes, poly(amino acids), PVP, polysaccharides, proteins, or polynucleotides.

In an embodiment of the present disclosure, the hormone is a reproductive hormone selected from follicle stimulating hormone (FSH), luteinizing hormone (LH), gonadotropin releasing hormone (GnRH), progesterone, human chorionic gonadotropin (hCG), estrogen, or testosterone.

In an embodiment of the present disclosure, the microneedle patch is used for administration of 10-100 IU of the hormone.

In an embodiment of the present disclosure, the microneedle patch is used for administration of 40-60 IU of the hormone.

In an embodiment of the present disclosure, the microneedle patch is configured for replacement daily over 2-14 days.

In an embodiment of the present disclosure, the microneedle patch is for use with at least one intradermal or subdermal injection of at least one additional large molecule therapeutic.

In another aspect, a method of manufacturing a dissolvable microneedle patch for intradermal administration of a hormone to a subject is disclosed. The method includes a first step of preparing a matrix material. The matrix material is prepared by mixing non-crosslinked high molecular weight hyaluronic acid, methylcellulose, and at least one large molecule therapeutic with heated water to form a hydrated matrix material. The method includes a second step of cooling the hydrated matrix material to room temperature. Further, the method includes a third step of casting the cooled matrix material into a microneedle mold and drying the matrix material within the microneedle mold to form a plurality of microneedles. The plurality of microneedles extend from a base layer of the microneedle patch.

In yet another aspect, a method of delivering a hormone to a subject in need of the hormone is disclosed. The method includes a first step of applying a dissolvable microneedle patch to a skin of the subject. The dissolvable microneedle patch includes a substantially planar base layer and a plurality of microneedles. The plurality of microneedles extend from a surface of the base layer. Each of the plurality of microneedles has a height of 1000-3000 μm. In addition, tach of the plurality of microneedles includes a solidified matrix. The solidified matrix includes 0.5-20% w/w non-crosslinked high molecular weight hyaluronic acid. The non-crosslinked high molecular weight hyaluronic acid has a molecular weight of 20-1000 kDa. In addition, the matrix includes 0.5-20% w/w methylcellulose. The methylcellulose has a viscosity of 100-8000 cP. Further, the matrix includes 0-20% w/w non-crosslinked low molecular weight hyaluronic acid. The non-crosslinked low molecular weight hyaluronic acid has a molecular weight of 20-1000 kDa. The matrix includes the hormone and no added chemical crosslinking agents. The method includes a second step of exerting sufficient force on the microneedle patch to cause the plurality of microneedles to penetrate the skin and enter intradermal tissue of the subject. Further, the method includes a third step of maintaining the microneedle patch in contact with the skin until at least a portion of the solidified matrix dissolves. Moreover, the method include a fourth steo of removing the microneedle patch from the skin. Less than 100% of the hormone is encapsulated in a biodegradable polymer. The plurality of microneedles dissolve within 1-14 days following insertion and the biodegradable polymer degrades within 1-14 days following insertion.

### BRIEF DESCRIPTION OF DRAWINGS

For a better understanding of the various described embodiments, reference should be made to the Detailed Description below, in conjunction with the following drawings in which like reference numerals refer to corresponding parts throughout the figures.

FIG. 1 illustrates an overall layout of a dissolvable microneedle patch, in accordance with various embodiments of the present disclosure;

FIG. 2 illustrates a flowchart of a method for manufacturing the dissolvable microneedle patch of FIG. 1, in accordance with an embodiment of the present disclosure;

FIG. 3 illustrates a flowchart of a method for delivering a hormone to a subject in need using the dissolvable microneedle patch of FIG. 1, in accordance with an embodiment of the present disclosure;

FIG. 4 illustrates a schematic representation of dimensional parameters of the plurality of microneedles including microneedle height, base width, and pitch, in accordance with various embodiments of the present disclosure;

FIG. 5 illustrates a cross-sectional view of a microneedle structure showing distribution of at least one large molecule therapeutic within a microneedle matrix, in accordance with various embodiments of the present disclosure; and

FIG. 6 illustrates a schematic representation of dissolution and therapeutic release of the microneedles following insertion into skin layers, in accordance with various embodiments of the present disclosure.

In accordance with common practice, the various features illustrated in the drawings may not be drawn to scale. Accordingly, the dimensions of the various features may be arbitrarily expanded or reduced for clarity. In addition, some drawings may not depict all the components of a given system, method, or device. Finally, like reference numerals may be used to denote features throughout the specification and figures.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limited to the disclosure. All publications, patent applications, patents, figures and other references mentioned herein are expressly incorporated by reference in their entirety.

The present disclosure is not limited to the particular systems, devices and methods described, as these may vary. The terminology used in the description is for the purpose of describing the particular versions or embodiments only and is not intended to limit the scope. Such aspects of the disclosure may be embodied in many different forms. These embodiments are provided so that this disclosure will be thorough and complete and will fully convey its scope to those skilled in the art.

The present disclosure is not limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and it is not intended to be limiting.

As used in this document, the singular forms “a”, “an” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in term of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

It will be understood by those within the art that, in general, terms used herein, and especially in the appended claims (for example, bodies of the appended claims) are generally intended as “open” terms (for example, the term “including” should be interpreted as “including but not limited to”, the term “having” should be interpreted as “having at least”, the term “includes” should be interpreted as “includes but is not limited to”, etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a”and/or “an”should be interpreted to mean “at least one”or “one or more”) ; the same holds true for the use of definite articles used to introduce claim restrictions. In addition, even if a specific number of an introduced claim recitation is explicitly recited, those skilled in the art will recognize that such recitation should be interpreted to mean at least the recited number (for example, the bare recitation of “two recitations”, without other modifiers means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc. It will further be understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”

All percentages, parts and ratios are based upon the total weight of the compositions and all measurements made are at about 25° C., unless otherwise specified.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49”, “about 50”, “about 55”, means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The term “patient” and “subject” are interchangeable and may be taken to mean any living organism which may be treated with microneedle arrays and therapeutics of the present disclosure. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “animal” as used herein includes, but is not limited to, humans and non-human vertebrates such as wild, domestic, and farm animals.

The term “tissue” refers to any aggregation of similarly specialized cells which are united in the performance of a particular function.

The term “disorder” is used in this disclosure to mean, and is used interchangeably with, the terms disease, condition, or illness, unless otherwise indicated.

The terms “administer,” “administering” or “administration” as used herein refer to either directly administering a compound (also referred to as an agent of interest) or pharmaceutically acceptable salt of the compound (agent of interest) or a composition to a subject.

The term “treat,” “treated,” or “treating” as used herein refers to both therapeutic treatment, wherein the object is to reduce the frequency of, or delay the onset of, symptoms of a medical condition, enhance the texture, appearance, color, sensation, or hydration of the intended tissue treatment area of the tissue surface in a subject relative to a subject not receiving the compound or composition, or to otherwise obtain beneficial or desired clinical results. For the purposes of this invention, beneficial or desired clinical results include, but are not limited to, reversal, reduction, or alleviation of symptoms of a condition; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

In some embodiments, the compounds and methods disclosed herein can be utilized with or on a subject in need of such treatment, which can also be referred to as “in need thereof.” As used herein, the phrase “in need thereof” means that the subject has been identified as having a need for the particular method or treatment and that the treatment has been given to the subject for that particular purpose.

As used herein, the term “therapeutic” or “therapeutic agent” or “pharmaceutically active agent” means an agent utilized to treat, combat, ameliorate, prevent or improve an unwanted condition or disease of a patient.

The term “composition” as used herein refers to a combination or a mixture of two or more different ingredients, components, or substances.

The term “skin” as used herein refers to the thin layer of tissue forming the natural outer covering of the body of a person or animal. The skin is made of the epidermis and dermis. As used herein, the stratum corneum is the outermost layer of the epidermis and the papillary dermis is the uppermost layer of the dermis.

The term “immediate release” as used herein refers to polymers designed to release at least 75% of the therapeutically active ingredients on a timescale of about 0 hours to about 24 hours.

The term “sustained release” as used herein refers to polymers that do not release more than 75% of the therapeutically active ingredients until a time greater than about 24 hours after application.

The term “biodegradable polymer” as used herein refers to a polymer that breaks down or dissolved after introduction to the skin and release of the therapeutically active ingredient.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

The present disclosure relates to dissolvable microneedle patch technologies configured for administration of therapeutically active agents to a subject through skin-based delivery pathways. The dissolvable microneedle patch includes a wearable medical device configured to deliver large molecule therapeutics using minimally invasive intradermal administration.

Administration of therapeutic agents through skin-based pathways presents technical challenges for therapeutics having large molecular size and structural complexity. A microneedle patch includes microscale projections arranged on a support structure. The microscale projections penetrate the stratum corneum and access intradermal tissue regions. The microscale projections avoid stimulation of deeper nerve structures associated with conventional hypodermic injections. Intradermal placement allows therapeutic agents to enter vascularized and lymphatic environments that support systemic absorption. Intradermal administration reduces discomfort associated with conventional needle injection.

The large molecule therapeutics include biologically derived compounds having high molecular weight and structural sensitivity. The large molecule therapeutics include proteins, peptides, antibodies, nucleic acids, and hormone-based therapeutics used in endocrine regulation, fertility treatment, metabolic therapy, and immunological applications. Therapeutic effectiveness of the large molecule therapeutics depends upon delivery of intact molecules into viable tissue regions that support the systemic absorption and bioavailability.

Skin functions as a biological barrier that restricts transport of hydrophilic and high molecular weight compounds. Conventional passive transdermal delivery systems rely upon diffusion across the stratum corneum. The passive transdermal delivery systems remain suitable primarily for small, lipophilic therapeutic agents. In addition, the passive transdermal delivery systems do not transport intact large molecule therapeutics into viable tissue due to molecular size and permeability limitations. Clinical administration of large molecule therapeutics relies predominantly upon subcutaneous injection or intramuscular injection. Injection-based administration introduces procedural discomfort and needle-associated anxiety. Repeated bolus dosing introduces variability in therapeutic exposure. Hormone therapies and biologic regimens often require repeated injections, which increase administration burden and reduce compliance.

The microneedle delivery systems form micron-scale pathways across the skin barrier to enable the intradermal administration. The dissolving microneedle technologies incorporate the therapeutic agents within dissolvable matrix materials. Dissolving formats eliminate sharps waste and avoid retained delivery hardware. Early dissolving microneedle systems focus on delivery of small-molecule drugs or low-dose biologic agents having limited payload requirements.

The delivery of the large molecule therapeutics using the dissolving microneedles introduces competing structural and material constraints. A microneedle intended for administration requires mechanical rigidity sufficient to penetrate skin without bending, buckling, or fracturing during application. Also, the dissolving microneedle requires solubility sufficient to permit dissolution after insertion for therapeutic release. Materials that preserve the large molecule stability are hydrophilic and readily soluble. Increased solubility reduces achievable mechanical strength during insertion. Increasing solids content or polymer density improves rigidity but alters dissolution behavior and may reduce compatibility with the large molecule therapeutics.

Further, the large molecule therapeutics impose drug loading constraints that approach physical limits of conventional dissolving microneedle patches. Many therapeutic regimens require delivery of relatively high drug quantities or repeated administration to maintain therapeutic exposure. Increased therapeutic loading within the dissolving microneedles disrupts structural continuity of matrix materials. Structural disruption produces brittle architectures, deformation during insertion, premature hydration, or loss of mechanical integrity before successful penetration. Limited volumetric capacity of individual microneedles restricts achievable payload and contributes to inconsistent dosing performance.

Certain sustained-delivery microneedle approaches rely upon retained structures. The structures include non-dissolving microneedles, detachable microneedle tips, hollow microneedles coupled to reservoirs, or implanted depot materials remaining within skin after administration. The retained materials introduce variability in the release behavior and complicate repeat administration protocols requiring predictable dosing intervals.

Delivery reliability across a microneedle array presents an additional design consideration. Individual microneedles within the array may exhibit variable insertion depth due to geometric variation, material properties, or application force variation. Partial insertion across the array produces heterogeneous intradermal deposition of therapeutic agents and reduces dose consistency. The large molecule therapeutics require consistent intradermal placement to achieve controlled exposure.

Certain dissolving microneedle designs link insertion mechanics and therapeutic release behavior to identical material parameters. Increasing structural rigidity to improve insertion performance alters dissolution characteristics and release kinetics. Modifying the dissolution behavior to extend therapeutic exposure reduces mechanical integrity during application. Coupling between mechanical performance and release behavior limits adaptability for administration of the large molecule therapeutics requiring controlled exposure profiles.

Manufacturing and reproducibility considerations influence scalability of the dissolving microneedle patches. Variations in formulation behavior, mold filling, drying conditions, or material concentration influence microneedle geometry and structural integrity. Processing variation produces incomplete microneedle formation, brittleness, structural defects, or non-uniform arrays. Large molecule formulations increase sensitivity to fabrication parameters due to higher solids loading requirements and spatial distribution constraints within microneedle structures.

The present disclosure provides a dissolvable microneedle patch configured to enable the intradermal administration of the large molecule therapeutics. The dissolvable microneedle patch facilitates delivery across the skin barrier into intradermal tissue regions that support systemic availability of biologically active agents. The dissolvable microneedle patch administers therapeutically effective quantities of the large molecule therapeutics and preserve molecular integrity during administration. Delivery occurs without chemical modification, conjugation, or structural alteration of the therapeutic molecule. Administration uses native therapeutic formulations employed in established clinical treatment protocols.

The dissolvable microneedle patch enables controlled therapeutic exposure through intradermal placement of the large molecule therapeutics without reliance on subcutaneous or intramuscular injection. The intradermal administration reduces variability associated with repeated bolus injection and supports maintenance of therapeutic levels over defined treatment intervals used in hormone-based and biologic regimens.

The dissolvable microneedle patch operates without retained implantable components, non-dissolving microneedles, detachable microneedle tips, or depot structures remaining within skin after administration. The microneedles dissolve after insertion and do not leave residual hardware within tissue. Repeat administration proceeds without residual material accumulation.

Embodiments of the present disclosure support administration of reproductive hormones and other biologically active large molecule therapeutics that require repeat dosing within defined treatment intervals. Administration using the dissolvable microneedle patch reduces dependence on repeated injections and supports predictable therapeutic exposure consistent with clinical treatment protocols.

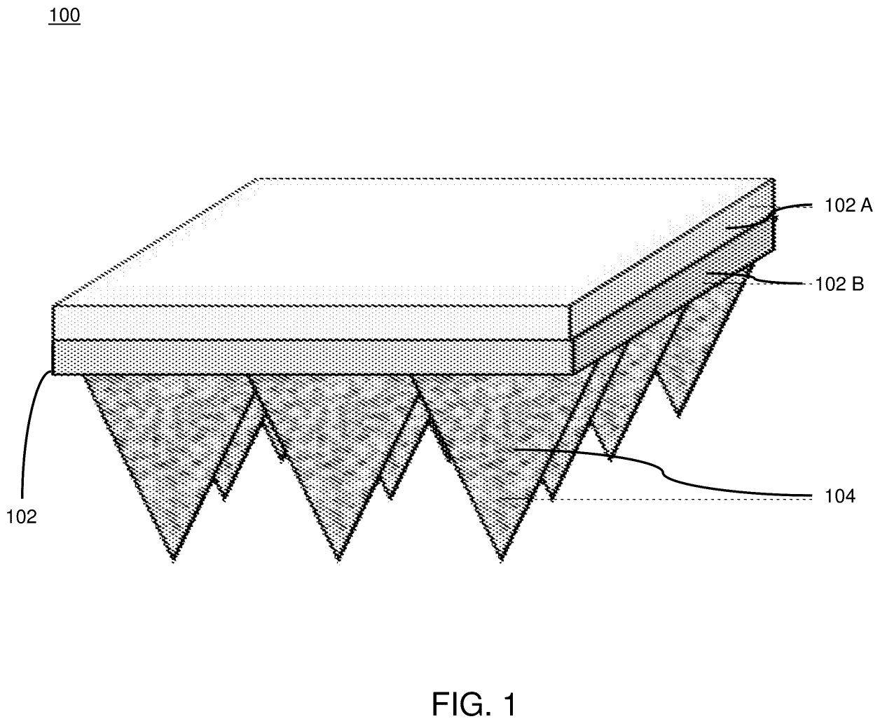

FIG. 1 illustrates an overall layout of a dissolvable microneedle patch 100, in accordance with various embodiments of the present disclosure. The dissolvable microneedle patch 100 includes a wearable drug delivery device configured for intradermal administration of at least one large molecule therapeutic to a subject. The dissolvable microneedle patch 100 defines a skin-contacting administration platform configured for placement on an external body surface of a subject. The external body surface includes an upper arm region, an abdominal region, or another anatomically suitable administration site. The dissolvable microneedle patch 100 includes a base layer 102 and a plurality of microneedles 104. The plurality of microneedles 104 extend outwardly from a skin-facing surface of the base layer 102.

The base layer 102 includes a substantially planar support structure configured to maintain spatial arrangement and mechanical alignment of the plurality of microneedles 104 during application. The base layer 102 transmits externally applied force toward the plurality of microneedles 104 to support penetration of skin during administration. The base layer 102 maintains positional stability of the plurality of microneedles 104 relative to a skin surface during placement of the dissolvable microneedle patch 100. The base layer 102 operates as a structural carrier that supports handling, transportation, storage, and controlled deployment of the dissolvable microneedle patch 100 prior to administration. The base layer 102 distributes externally applied mechanical force substantially uniformly across the plurality of microneedles 104 during application. Uniform distribution reduces localized stress concentrations that otherwise cause deformation or incomplete engagement of individual microneedles within the microneedle array.

In certain embodiments, the base layer 102 exhibits flexibility sufficient to accommodate curvature and movement of skin and simultaneously maintain positional alignment of the plurality of microneedles 104. The base layer 102 maintains attachment during subject movement following placement of the dissolvable microneedle patch 100. In an embodiment of the present disclosure, the base layer 102 has a surface area of approximately 4 cm×4 cm, 3 cm×3 cm, or 1.5 cm×1.5 cm.

The base layer 102 includes a non-adhesive top layer 102A positioned opposite to the plurality of microneedles 104. The non-adhesive top layer 102A provides a handling surface configured to permit manual or assisted application of the dissolvable microneedle patch 100 by a user or clinician. The non-adhesive top layer 102A protects structural components of the dissolvable microneedle patch 100 from environmental exposure prior to administration. The non-adhesive top layer 102A reduces unintended adhesion to external surfaces during storage and handling and supports controlled application force distribution across the dissolvable microneedle patch 100.

Further, the non-adhesive top layer 102A provides an externally exposed surface configured to receive manual pressure during application. The non-adhesive top layer 102A enables a user to apply compressive force using fingers or an applicator device without adherence of the upper surface to the user. Further, the non-adhesive top layer 102A protects internal structural elements of the dissolvable microneedle patch 100 from environmental exposure prior to administration.

Furthermore, the base layer 102 includes an adhesive surface 102B arranged along a skin-facing region of the base layer 102. The adhesive surface 102B secures the dissolvable microneedle patch 100 to skin following placement. The adhesive surface 102B forms a perimeter or partial perimeter surrounding a region from which the plurality of microneedles 104 extend. Peripheral adhesion retains the dissolvable microneedle patch 100 in position without obstructing engagement of the plurality of microneedles 104 with skin. The adhesive surface 102B maintains contact between the dissolvable microneedle patch 100 and skin during the administration period. The adhesive surface 102B reduces lateral displacement or lifting of the dissolvable microneedle patch 100 during the administration period. Stabilization provided by the adhesive surface 102B supports uniform engagement of substantially all microneedles within the plurality of microneedles 104 across the application region.

The adhesive surface 102B reduces lateral displacement or lifting of the dissolvable microneedle patch 100 during administration. Stabilization provided by the adhesive surface 102B supports engagement of substantially all microneedles within the plurality of microneedles 104 across the application region.

The base layer 102 includes materials that exhibit flexibility sufficient to conform to natural contours of a body surface and maintain structural support required for administration. The base layer 102 remains externally removable after administration and does not remain implanted within tissue.

In certain embodiments, the base layer 102 includes polymeric materials compatible with medical patch applications. The base layer 102 optionally includes a matrix material shared with the plurality of microneedles 104. The plurality of microneedles 104 form congruous extensions of the base layer 102. Structural continuity between the base layer 102 and the plurality of microneedles 104 supports mechanical stability during application.

The plurality of microneedles 104 are arranged across the surface of the base layer 102 in an array configuration. The plurality of microneedles 104 extend away from the base layer 102 in a direction configured for insertion into skin during administration. The plurality of microneedles 104 are distributed across the base layer 102 to enable administration of therapeutically effective quantities of the therapeutic agent through collective action of multiple microneedles rather than reliance on a single penetration element.

The dissolvable microneedle patch 100 includes greater than five microneedles and may include between approximately 50-1000 microneedles distributed across the base layer 102. Distribution of the plurality of microneedles 104 across the base layer 102 supports force sharing during application and reduces dependence on localized pressure during insertion.

The plurality of microneedles 104 are arranged in a predefined geometric array extending from the base layer 102. Array spacing between adjacent microneedles is selected to balance delivery density with mechanical independence of individual microneedles during insertion. Separation between adjacent microneedles reduces risk of collective buckling or interference during application.

Each microneedle of the plurality of microneedles 104 includes a solid structure extending from the base layer 102. Each microneedle of the plurality of microneedles 104 are dimensioned to penetrate through an outer skin barrier while avoiding interaction with deeper nerve-rich regions, thereby enabling minimally invasive administration. Each microneedle of the plurality of microneedles 104 includes a tapered configuration terminating in a tip region having a contact area smaller than a contact area of a 30-gauge hypodermic needle.

The plurality of microneedles 104 include microneedles having substantially equal lengths or approximately equal lengths across the array to promote uniform interaction with skin during administration. In certain embodiments, the plurality of microneedles 104 include microneedles having at least two different lengths arranged across the base layer 102.

Each microneedle of the plurality of microneedles 104 includes a height configured to terminate within dermal tissue following insertion. Microneedle height is selected within a range sufficient to bypass a stratum corneum and epidermal barrier while avoiding deep tissue penetration associated with conventional injection needles.

The plurality of microneedles 104 may include conical, pyramidal, square pyramidal, trigonal pyramidal, hexagonal pyramidal, heptagonal pyramidal, octagonal pyramidal, nonagonal pyramidal, or decagonal geometries. Selection of microneedle geometry supports mechanical robustness during insertion while maintaining compatibility with dissolvable material construction.

Each microneedle of the plurality of microneedles 104 includes a tip portion representing approximately five percent to ninety-five percent of a total microneedle volume. In certain embodiments, the plurality of microneedles 104 are boreless and do not include hollow channels for fluid injection.

In certain embodiments, each microneedle of the plurality of microneedles 104 contains an equal or approximately equal quantity of a therapeutic agent to promote dose uniformity across the dissolvable microneedle patch 100. In alternative embodiments, individual microneedles contain unequal quantities of the therapeutic agent to enable controlled distribution across the dissolvable microneedle patch 100.

In an embodiment of the present disclosure, the dissolvable microneedle patch 100 optionally includes at least one sensor integrated within the base layer 102. The sensor is positioned within or coupled to the base layer 102 such that sensing functionality is achieved while maintaining structural integrity and wearability of the dissolvable microneedle patch 100 during administration.

In certain embodiments, the sensor includes at least one electrochemical sensor, optical sensor, biosensor, impedance-based sensor, thermal sensor, or microfluidic sensing element configured to detect one or more biological analytes or physiological parameters associated with therapeutic administration. Further, the sensor may include signal acquisition circuitry, wireless communication components, or data processing elements configured to transmit or process detected physiological information during or after administration of the dissolvable microneedle patch.

The sensor is configured to detect or measure concentration levels of at least one hormone or biological marker present within a bodily fluid of the subject. In an embodiments, the sensor detects hormone concentrations associated with therapeutic administration to enable monitoring of physiological response following application of the dissolvable microneedle patch 100. Detection of hormone levels enables assessment of whether therapeutically relevant exposure levels are achieved or maintained during an administration interval.

In certain other embodiments, the sensor detects biological parameters present within blood, interstitial fluid, or sweat accessible near a skin interface underlying the dissolvable microneedle patch 100. The sensor is configured to obtain biochemical or physiological information without requiring removal of the dissolvable microneedle patch 100 from the skin surface. Monitoring of interstitial fluid parameters enables indirect evaluation of systemic biomarker levels. The evaluation results from physiological correlation between interstitial fluid composition and circulating biological analytes.

The sensor optionally detects temperature associated with interstitial fluid or localized skin regions underlying the dissolvable microneedle patch 100. Temperature detection supports monitoring of localized physiological conditions during administration, including assessment of skin response, metabolic activity, or environmental influences that may affect therapeutic administration performance. The sensor operates without interfering with dissolution of the plurality of microneedles and remains removable together with the base layer following completion of administration.

In certain embodiments, the sensor operates continuously or intermittently during an administration period to enable real-time or periodic monitoring of biological conditions associated with therapeutic delivery. Integration of the sensor within the base layer 102 enables combined therapeutic administration and physiological monitoring within a single wearable platform while maintaining removability of the dissolvable microneedle patch 100 following use.

The structural configuration illustrated in FIG. 1 enables application of the dissolvable microneedle patch 100 onto a skin surface using manual pressure sufficient to position the plurality of microneedles 104 for intradermal administration while maintaining user comfort and positional stability during use.

In an embodiment of the present disclosure, the dissolvable microneedle patch 100 includes structural and functional layers formed from biocompatible materials suitable for dermal contact and medical administration applications.

In certain embodiments, the base layer 102 includes a flexible polymeric substrate configured to provide mechanical support to the plurality of microneedles 104 during handling and application. The base layer 102 includes biocompatible polymer materials capable of maintaining dimensional stability while conforming to contours of a skin surface. The base layer 102 optionally includes medical-grade polymer films selected from polyurethane, polyethylene terephthalate (PET), polyethylene, polypropylene, silicone-based elastomers, thermoplastic elastomers, or combinations thereof. The base layer 102 optionally includes a biodegradable or water-compatible polymer matrix consistent with materials used in formation of the plurality of microneedles 104 such that the plurality of microneedles 104 form congruous extensions of the base layer 102.

In certain embodiments, the non-adhesive top layer 102A includes a protective backing material configured to permit manual handling and application of the dissolvable microneedle patch 100 without adherence to external surfaces. The non-adhesive top layer 102A includes a non-tacky, moisture-resistant material selected from polymer films, laminated medical backings, coated polymer sheets, or elastomeric films. The non-adhesive top layer 102A include but are not limited to polyurethane film, polyester film, polyethylene film, silicone-coated polymer layers, or multilayer laminate structures configured to distribute externally applied force across the base layer 102 during administration.

In certain embodiments, the adhesive surface 102B includes a skin-compatible adhesive configured to temporarily secure the dissolvable microneedle patch 100 onto a skin surface throughout an administration interval. The adhesive surface 102B includes pressure-sensitive medical adhesives selected from acrylic-based adhesives, silicone-based adhesives, hydrocolloid adhesives, hydrogel adhesives, rubber-based medical adhesives, or combinations thereof. The adhesive surface 102B maintains attachment while permitting atraumatic removal of the dissolvable microneedle patch 100 following administration without substantial skin irritation or residue formation.

In certain embodiments, the plurality of microneedles 104 include dissolvable biocompatible polymer materials configured to maintain mechanical integrity during insertion and subsequently dissolve following administration. The plurality of microneedles 104 include a matrix material including high molecular weight (HMW), non-crosslinked hyaluronic acid and methylcellulose. The matrix material optionally includes low molecular weight (LMW) hyaluronic acid to adjust mechanical and material properties of the plurality of microneedles 104.

In some embodiments, the solidified matrix, the plurality of microneedles 104 and/or the encapsulated large molecule therapeutic may include one or more pharmaceutically acceptable carriers (excipients). In some embodiments, the excipient may be selected from at least one of the following: lactose, sucrose, glucose, mannitol, sorbitol, trehalose, fructose, galactose, dextrose, xylitol, maltitol, raffinose, dextran, maltodextrin, cyclodextrin, collagen, glycine, histidine, calcium carbonate, magnesium stearate, serum albumin (human and/or animal sources), gelatin, chitosan, DNA, hyaluronic acid, polyvinylpyrrolidone, polyvinyl alcohol, polylactic acid (PLA), polyglycolic acid (PGA), polyactive co-glycolic acid (PLGA), polyethylene glycol (PEG, PEG300, PEG400, PEG600,PEG3350, PEG4000), cellulose, methylcellulose, carboxymethyl cellulose, sodium carboxymethyl cellulose, hydroxypropyl methylcellulose, acacia, Lecithin, Polysorbate 20, Polysorbate 80, Pluronic F-68, Sorbitantrioleate (span 85), EDTA, hydroxypropyl cellulose, sodium chloride, sodium phosphate, ammonium acetate, potassium phosphate, sodium citrate, sodium hydroxide, sodium carbonate, Tris base-65, Tris acetate, Tris HCl-65, citrate buffer, talc, silica, fats, methyl paraben, propyl paraben, selenium, vitamins (A, E, C, retinyl palmitate, and selenium), amino acids (methionine, cysteine, arginine), citric acid, sodium citrate, benzyl alcohol, chlorbutanol, cresol, phenol, thimerosal, acetone, sodium bisulfate, ascorbyl palmitate, ascorbate, castor oil, cottonseed oil, alum, aluminum hydroxide, aluminum phosphate, calcium phosphate hydroxide, paraffin oil, squalene, Quil A, IL-1, IL-2, IL-12, Freund's complete adjuvant, Freund's incomplete adjuvant, killed Bordetella pertussis, Mycobacterium bovis, and toxoids. In some embodiments, an excipient may be a polymer. In some embodiments, an excipient may be biodegradable polymer. In some embodiments, an excipient is a polymer, wherein the polymer is maltodextrin. In some embodiments, there is one excipient. In some embodiments, there are two excipients. In some embodiments, there are three or more excipients. In some embodiments, the two excipients are maltodextrin and lactose, maltodextrin and sodium thiosulfate, maltodextrin and trehalose, maltodextrin and sodium citrate, maltodextrin and sorbitol, maltodextrin and arginine, maltodextrin and arginine, arginine and lactose, arginine and sodium thiosulfate, arginine and trehalose, arginine and sodium citrate, arginine and sorbitol, sorbitol and lactose, sorbitol and sodium thiosulfate, sorbitol and trehalose, sorbitol and sodium citrate, sodium citrate and lactose, sodium citrate and sodium thiosulfate, sodium citrate and trehalose, trehalose and lactose, trehalose and sodium thiosulfate, or sodium thiosulfate and lactose.

The materials forming the plurality of microneedles 104 are selected such that the microneedles retain sufficient rigidity for skin penetration while remaining capable of dissolution following administration. In certain embodiments of the present disclosure, mechanical strength sufficient to enable penetration of skin by the plurality of microneedles is achieved. The mechanical strength results from physical interactions present within the non-crosslinked polymer matrix forming each microneedle. The matrix material including high molecular weight hyaluronic acid and methylcellulose. The materials form an interconnected physical network resulting from polymer chain entanglement and intermolecular hydrogen bonding between adjacent polymer chains. The high molecular weight polymer chains establish resistance to compressive deformation and buckling during application. The hydrogen bonding interactions between hydroxyl and carboxyl functional groups provide temporary structural cohesion within the microneedle body. The combined effect of polymer chain entanglement and reversible intermolecular bonding enables the microneedles to maintain sufficient rigidity for intradermal penetration under externally applied force. The polymer chain entanglement and reversible intermolecular bonding preserves full dissolvability following exposure to physiological fluid after insertion. Upon hydration within skin tissue, disruption of hydrogen bonding interactions permits progressive dissolution of the non-crosslinked matrix without retention of rigid structural material within the subject. Also, the material selection supports compatibility with incorporation of at least one large molecule therapeutic without chemical modification of the therapeutic agent.

Accordingly, materials of the base layer 102, non-adhesive top layer 102A, adhesive surface 102B, and plurality of microneedles 104 collectively enable formation of a wearable dissolvable microneedle patch 100 suitable for intradermal administration while maintaining mechanical stability, biocompatibility, and user comfort during application and removal.

Each microneedle of the plurality of microneedles 104 includes the solidified matrix. The solidified matrix includes non-crosslinked high molecular weight hyaluronic acid from 0.5% to 20% by weight. The non-crosslinked high molecular weight hyaluronic acid has a molecular weight of 1000-1400 kDa. In addition, the solidified matrix includes from 0.5% to 20% by weight methylcellulose. The methylcellulose has a viscosity in a range of 100-8000 centipoise. The solidified matrix may further include from 0% to 20% by weight non-crosslinked low molecular weight hyaluronic acid. The non-crosslinked low molecular weight hyaluronic acid has a molecular weight of 20-1000 kDa. In an embodiment of the present disclosure, the solidified matrix may include a different compositional range within physiologically compatible limits.

The solidified matrix excludes added chemical crosslinking agents. Mechanical strength sufficient to permit intradermal penetration results from physical polymer chain entanglement and reversible intermolecular hydrogen bonding interactions. The plurality of microneedles 104 achieve structural rigidity without covalent crosslink formation. Absence of the chemical crosslinking agents enables complete biodegradability and controlled dissolution of the plurality of microneedles 104 following administration. In an embodiment of the present disclosure, the solidified matrix excludes additional chemical curing components.

In an embodiment of the present disclosure, the solidified matrix includes the hormone distributed throughout the solidified matrix. Less than 100% of the hormone is encapsulated in the biodegradable polymer dispersed within the solidified matrix. A non-encapsulated fraction of the hormone is directly embedded within the solidified matrix. An encapsulated fraction of the hormone is contained within discrete biodegradable polymer domains dispersed throughout the solidified matrix. The biodegradable polymer domains define localized reservoirs that control delayed release. In an embodiment of the present disclosure, the encapsulated fraction and the non-encapsulated fraction may exist in a different proportional distribution.

The biodegradable polymer degrades following insertion of the plurality of microneedles 104 into intradermal tissue under physiological conditions. The degradation of the biodegradable polymer occurs over a period of 1-14 days following the insertion. The controlled degradation regulates release of the encapsulated fraction of the hormone. In an embodiment of the present disclosure, the biodegradable polymer may exhibit a different degradation timeframe within a therapeutically defined window.

The plurality of microneedles 104 dissolve within intradermal tissue over a period of 1-14 days following the insertion. Dissolution of the solidified matrix and degradation of the biodegradable polymer occur without leaving non-dissolving structural components within tissue. The coordinated dissolution and the biodegradable polymer degradation enable staged release of the hormone following the insertion. In an embodiment of the present disclosure, the plurality of microneedles 104 may dissolve within a different controlled time interval.

Further, each microneedle of the plurality of microneedles 104 has a height in a range of 1000-3000 μm. The height enables penetration beyond stratum corneum and epidermis and into dermal tissue and simultaneously help avoid deep subcutaneous penetration. In an embodiment of the present disclosure, each microneedle of the plurality of microneedles 104 may have a different height range within a clinically defined insertion envelope.

As used herein, the term “high molecular weight (HMW) hyaluronic acid” refers to hyaluronic acid characterized by a molecular weight in a range of 1000-1400 kilodaltons. In an embodiment, the low molecular weight (LMW) hyaluronic acid has a molecular weight of 20-300 kilodaltons. In an embodiment, a medium molecular weight (LMW) hyaluronic acid has a molecular weight of 300-1000 kilodaltons.

In an embodiment of the present disclosure, the solidified matrix includes the non-crosslinked high molecular weight hyaluronic acid from 1% to 10% by weight. In another embodiment of the present disclosure, the solidified matrix includes the non-crosslinked high molecular weight hyaluronic acid from 1% to 5% by weight. In yet another embodiment of the present disclosure, the solidified matrix includes 1% w/w of the non-crosslinked high molecular weight hyaluronic acid. In yet another embodiment of the present disclosure, the solidified matrix includes 2% w/w of the non-crosslinked high molecular weight hyaluronic acid. In yet another embodiment of the present disclosure, the solidified matrix includes an individual concentration of the non-crosslinked high molecular weight hyaluronic acid selected from 3%, 4%, 5%, 6%, 7%, 8%, 9%, or 10% by weight. In yet another embodiment of the present disclosure, the solidified matrix may include a different concentration of the high modecular weight hyaluronic acid within a therapeutically acceptable range.

In an embodiment of the present disclosure, the solidified matrix includes the non-crosslinked low molecular weight hyaluronic acid from 0.5% to 20% by weight. In another embodiment of the present disclosure, the solidified matrix includes the non-crosslinked low molecular weight hyaluronic acid from 1% to 10% by weight. In yet another embodiment of the present disclosure, the solidified matrix includes an individual concentration of the non-crosslinked high molecular weight hyaluronic acid selected from 1%, 2%, 3%, 4%, 5%, 6%, 7%, 8%, 9%, or 10% by weight. In yet another embodiment of the present disclosure, the solidified matrix includes a different low molecular weight hyaluronic acid concentration range.

In an embodiment of the present disclosure, the solidified matrix includes the methylcellulose with a viscosity of between 100 cP and 10,000 cP. In another embodiment of the present disclosure, the methylcellulose has a viscosity of 400 cP. In yet another embodiment of the present disclosure, the methylcellulose has a viscosity of 4000 cP. In yet another embodiment of the present disclosure, the methylcellulose may have a viscosity within a different operable range consistent with structural reinforcement requirements. In an embodiment of the present disclosure, the solidified matrix includes 0.5 to 20% w/w methylcellulose. In another embodiment of the present disclosure, the solidified matrix includes 1 to 10% w/w methylcellulose. In some embodiments, the solidified matrix may include 1%, 2%, 3%, 4%, 5%, 6%, 7%, 8%, 9% or 10% w/w methylcellulose.

In an embodiment of the present disclosure, the plurality of microneedles 104 are dimensioned to terminate within epidermal tissue rather than dermal tissue. Termination depth selection determines hormone deposition location within skin layers.

In an embodiment of the present disclosure, the biodegradable polymer includes a synthetic polymer. In another embodiment of the present disclosure, the biodegradable polymer includes a non-synthetic polymer selected from polysaccharides, proteins, or polynucleotides. In yet another embodiment of the present disclosure, the biodegradable polymer may include a different biodegradable material compatible with intradermal degradation. The biodegradable polymer domains are configured to degrade following insertion into intradermal tissue under physiological conditions. Degradation of the biodegradable polymer occurs over a period of approximately 1-14 days following insertion.

The plurality of microneedles 104 are configured to dissolve within intradermal tissue over a period of approximately 1-14 days following insertion. Dissolution of the microneedle matrix and degradation of the biodegradable polymer occur without leaving non-dissolving structural components within tissue. Coordinated dissolution and polymer degradation enable staged release of the hormone following insertion.

In an embodiment of the present disclosure, the plurality of microneedles 104 have a preferred height that lies within 1.7-2.3 mm. In another embodiment of the present disclosure, each microneedle of the plurality of microneedles 104 has a height of approximately 1 mm. In yet another embodiment of the present disclosure, each microneedle of the plurality of microneedles 104 has a height of approximately 3 mm. In yet another embodiment of the present disclosure, the plurality of microneedles 104 may have a different height range.

In an embodiment of the present disclosure, the plurality of microneedles 104 have a preferred base width that lies within 100-500 μm. In another embodiment of the present disclosure, the plurality of microneedles 104 may have a preferred base width up to 2 mm. In yet another embodiment of the present disclosure, the plurality of microneedles 104 may have a different base width range. In an embodiment of the present disclosure, the plurality of microneedles 104 have a preferred pitch that lies within 100-3000 μm. In another embodiment of the present disclosure, the plurality of microneedles 104 may have a different pitch range.

In an embodiment of the present disclosure, a combined solids content of the non-crosslinked high molecular weight hyaluronic acid, the optional non-crosslinked low molecular weight hyaluronic acid, and the methylcellulose within the solidified matrix forming each microneedle of the plurality of microneedles 104 lies within 2-20% w/w. In another embodiment of the present disclosure, the combined solids content lies within a broader operative range of 1-30% w/w. In an embodiment of the present disclosure, if the combined solids content falls below approximately 1% w/w, mechanical strength of each microneedle of the plurality of microneedles 104 decreases and risk of deformation during insertion increases. In another embodiment of the present disclosure, if the combined solids content exceeds approximately 30% w/w, brittleness of the solidified matrix increases and dissolution time may extend beyond an intended therapeutic window. In yet another embodiment of the present disclosure, the solidified matrix may have a different combined polymer solids content range.

In an embodiment of the present disclosure, the plurality of microneedles 104 dissolve within approximately 6-24 hours following the insertion. In another embodiment of the present disclosure, the plurality of microneedles 104 may have a different dissolution time range. In an embodiment of the present disclosure, release of the hormone from the dissolvable microneedle patch 100 includes immediate release behavior. The immediate release refers to release of at least 75% of the hormone within 0-24 hours following application of the dissolvable microneedle patch 100. In another embodiment of the present disclosure, the release of the hormone from the dissolvable microneedle patch 100 includes the sustained release behavior. The sustained release refers to release of less than 75% of the hormone within 24 hours following application of the dissolvable microneedle patch 100 with continued release over multiple days. In yet another embodiment of the present disclosure, release of a hormone includes a multi-phase release profile. The multi-phase release profile includes an initial release phase corresponding to dissolution of the non-encapsulated fraction of the hormone and a subsequent release phase corresponding to the degradation of the biodegradable polymer. The biodegradable polymer contains an encapsulated fraction of the hormone. In yet another embodiment of the present disclosure, the dissolvable microneedle patch 100 may provide a different therapeutic release profile.

In an embodiment of the present disclosure, the plurality of microneedles 104 deliver a large-molecule therapeutic. The large-molecule therapeutic has a molecular weight greater than approximately 1 kDa and up to approximately 200 kDa. In an embodiment of the present disclosure, the large-molecule therapeutic includes fertility hormones within a molecular weight range typical for peptide and protein hormones. In another embodiment of the present disclosure, the plurality of microneedles 104 may deliver the large-molecule therapeutic with a different molecular weight range.

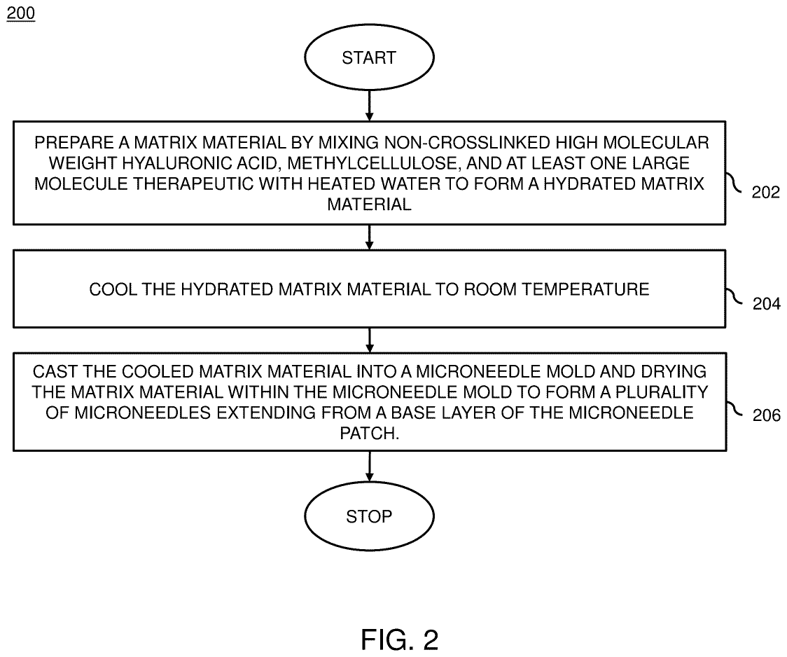

FIG. 2 illustrates a flowchart 200 representing a method of manufacturing the dissolvable microneedle patch 100 for the intradermal administration of the hormone to the subject, in accordance with various embodiments of the present disclosure. Flowchart 200 includes sequential processing stages that form base layer 102 and plurality of microneedles 104 from a matrix material.

The manufacturing method includes a first step 202 of preparing a matrix material. The preparation step 202 includes mixing the non-crosslinked high molecular weight hyaluronic acid, the methylcellulose, and at least one large molecule therapeutic with heated water to form a hydrated matrix material.

The term “matrix material” refers to a polymeric formulation that forms structural body of base layer 102 and the plurality of microneedles 104 after drying. The matrix material forms a physically entangled polymer network without chemical crosslinking agents. The term “non-crosslinked” refers to absence of covalent intermolecular crosslink bonds between polymer chains. Mechanical strength results from the polymer chain entanglement and hydrogen bonding interactions. The term “large molecule therapeutic” refers to a biologically active macromolecule. The large molecule therapeutic may include a protein, peptide, antibody, nucleic acid, or hormone. In certain embodiments, the at least one large molecule therapeutic includes the hormone for the intradermal administration.

The preparation step 202 includes hydrating polymer powders using heated water. In an embodiment, the heated water has a temperature above room temperature. In an embodiment, the heated water has a temperature within 20-80° C. In another embodiment of the present disclosure, the heated water has a temperature within 30-75° C. In yet another embodiment of the present disclosure, the heated water has a temperature of about 65° C. The heating enhances dissolution of the hyaluronic acid and the methylcellulose. In addition, the heating reduces polymer aggregation and promotes homogeneous dispersion.

In an embodiment of the present disclosure, the hydrated matrix material is formed through mechanical agitation. The mechanical agitation includes stirring, blending, vortexing, or controlled mixing using laboratory mixers.

In certain embodiments, the at least one large molecule therapeutic undergoes encapsulation within the biodegradable polymer prior to mixing. The term “encapsulation” refers to formation of polymeric particles that surround and entrap the therapeutic. The encapsulation may include formation of microspheres, nanospheres, microparticles, nanoparticles, or polymer-coated droplets. In an embodiment, the biodegradable polymer includes polyethylene glycol (PEG), polyvinyl alcohol (PVA), poly(lactic-co-glycolic acid) (PLGA), polycaprolactone (PCL), poly(lactic acid) (PLA), polyanhydride, poly(methyl methacrylate) (PMMA), poly(hydroxyethylmethacrylate) (PHEMA), polyurethane, poly(amino acid), polyvinylpyrrolidone (PVP), polysaccharide, protein, or polynucleotide-based materials. The encapsulation prior to the mixing creates dispersed therapeutic reservoirs within the hydrated matrix material. Further, the encapsulation supports sustained release after intradermal placement.

As used herein, the term “large molecule therapeutic” refers to complex biological compounds including proteins, antibodies, enzymes, growth factors, cytokines, nucleic acids, and peptides. Also, the large molecule therapeutics are commonly referred to a biologics. In some embodiments of the present disclosure, the large molecule therapeutic is a hormone, for example a peptide or protein hormone In some embodiments, the hormone is a reproductive or endocrine hormone. In some embodiments, the reproductive hormone is a hormone which possesses useful fertility stimulating or inhibiting properties and may be used in the treatment or prophylaxis of a disease or condition affecting the fertility of a female subject. In some embodiments, the reproductive hormone is for stimulating or inhibiting fertility in a female subject. In some embodiments, the hormone is progesterone, gonadotropin releasing hormone, follicle stimulating hormone (FSH), luteinizing hormone (LH), Human chorionic gonadotropin (hCG); estrogen, gonadotropin releasing hormone analogue (GnRH), a GnRH antagonist, GnRH agonist, or testosterone In some embodiments, the hormone is insulin or a glucagon-like peptide-1 (GLP-1) receptor agonist. In some embodiments, the large molecule therapeutic is a monoclonal antibody. In some embodiments, the antibody is Adalimumab (Humira), Etanercept (Enbrel), Etanercept (Enbrel), Dupilumab (Dupixent) or Teprotumumab (Tepezza). In some embodiments, the large molecule therapeutic is a growth factor or cytokine. In some embodiments, the large molecule therapeutic is interferon alpha/beta, interleukin-2 (IL-2) or erythropoietin (EPO, Epogen, Procrit).

In some embodiments, the large molecule therapeutic is an enzyme. In some embodiments, the large molecule therapeutic is lmiglucerase (Cerezyme), ldursulfase (Elaprase) or Alglucosidase alfa (Myozyme).

The manufacturing method includes a second step 204 of cooling the hydrated matrix material to room temperature. The cooling stabilizes the polymer chain entanglement and increases the viscosity. Further, the cooling includes controlled exposure to ambient temperature. The cooling includes temperature reduction from elevated hydration temperature to room temperature.

In certain embodiments, the cooling step 204 includes gently shaking the hydrated matrix material. The term “gently shaking” refers to low-amplitude oscillatory motion that maintains uniform dispersion without introducing air entrainment. The gentle shaking prevents sedimentation of encapsulated domains.

The manufacturing method includes a third step 206 of casting the cooled matrix material into a microneedle mold. The term “casting” refers to transferring viscous matrix material into micro-scale cavities of a mold that define geometry of the plurality of microneedles 104 and base layer 102. The microneedle mold includes a negative template that contains recessed cavities corresponding to microneedle dimensions. Microneedle mold fabrication includes micro-machining, lithography, laser ablation, micro-molding, or precision mechanical drilling. The casting includes gravitational filling or pressure-assisted filling.

In certain embodiments, the casting includes dispensing the matrix material using a syringe applicator. The term “syringe applicator” refers to a manually or mechanically actuated dispensing device that allows controlled volumetric delivery of viscous material. The syringe applicator enhances uniform filling of the microneedle mold cavities.

In certain embodiments, the casting step 206 further includes centrifugation of microneedle mold prior to drying. The term “centrifugation” refers to application of rotational force to drive matrix material into micro-scale cavities and remove entrapped air bubbles. The centrifugation increases density uniformity within the plurality of microneedles 104.

The manufacturing method includes a drying step 208 following the casting. The drying removes water from the matrix material and transforms the hydrated matrix into a solidified structure. In an embodiment of the present disclosure, the drying includes air drying for 24-48 hours. The term “air drying” refers to solvent evaporation under ambient or controlled environmental conditions. In another embodiment of the present disclosure, the drying includes freeze-drying. The term “freeze-drying” refers to freezing the matrix material followed by sublimation under reduced pressure. The freeze-drying preserves structural integrity of thermally sensitive hormones. Further, the drying step 208 forms the plurality of microneedles 104 extending from the base layer 102.

The drying establishes rigidity through the polymer chain entanglement and hydrogen bonding. The hydrogen bonding includes intermolecular attraction between hydroxyl and carboxyl functional groups present in the hyaluronic acid and the methylcellulose. The hydrogen bonding creates reversible cohesive interactions. The polymer chain entanglement includes physical interlacing of long-chain macromolecules. In addition, the polymer chain entanglement provides compressive strength without covalent crosslinking.

After drying, a manufacturer removes the dissolvable microneedle patch 100 from the microneedle mold. The removal of the dissolvable microneedle patch 100 yields the base layer 102 and the plurality of microneedles 104 as an integral structure.

The flowchart 200 may include additional preparatory or post-processing stages. Additional stages may include filtration, sterilization, degassing, pH adjustment, vacuum treatment, humidity control, packaging, dimensional verification, or mechanical testing. Sequence order may vary based on manufacturing optimization. In an embodiment of the present disclosure, the manufacturing method includes additional processing variations within disclosed material and temperature ranges. The manufacturer may adjust the hydration duration, the cooling duration, the centrifugal force magnitude, or the drying environment without departing from disclosed framework.

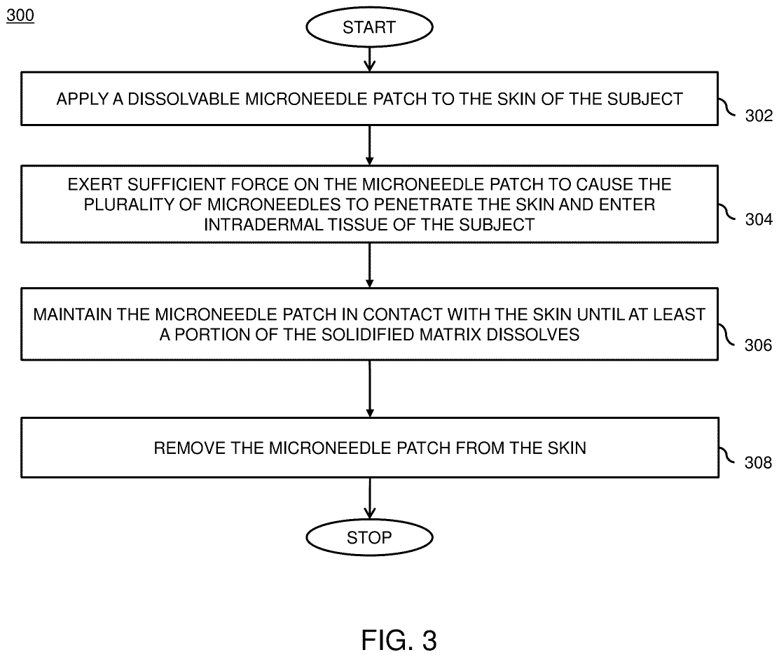

FIG. 3 illustrates a flowchart 300 representing a method of delivering the hormone to a subject in need thereof using the dissolvable microneedle patch 100, in accordance with various embodiments of the present disclosure. The flowchart 300 includes sequential administration stages that achieve intradermal placement of the hormone through the plurality of microneedles 104. The plurality of microneedles 104 extend from the base layer 102.

The method includes a first step 302 of applying the dissolvable microneedle patch 100 to a skin of the subject. The dissolvable microneedle patch 100 includes the substantially planar base layer 102 and the plurality of microneedles 104. The plurality of microneedles 104 extend from a surface of the base layer 102. Each microneedle of the plurality of microneedles 104 has a height of 1000-3000 μm (as explained already in the detailed description of FIG. 1). The solidified matrix refers to a dried polymer network forming structural body of the plurality of microneedles 104. The term “non-crosslinked” refers to absence of covalent intermolecular crosslinks between polymer chains. Mechanical rigidity of the solidified matrix results from the polymer chain entanglement and the hydrogen bonding. The term “hormone” refers to a biologically active endocrine signaling molecule suitable for intradermal administration.

The method includes a second step 304 of exerting sufficient force on the dissolvable microneedle patch 100 to cause the plurality of microneedles 104 to penetrate the skin and enter an intradermal tissue of the subject. The term “sufficient force” refers to a compressive force capable of overcoming mechanical resistance of stratum corneum and epidermis. The penetration places the plurality of microneedles 104 within the intradermal tissue. The term “intradermal tissue” refers to dermal region located beneath epidermis and above subcutaneous fat.

The method includes a third step 306 of maintaining the dissolvable microneedle patch 100 in contact with the skin until at least a portion of the solidified matrix dissolves. The plurality of microneedles 104 dissolve within 1-14 days following insertion.

The method includes a fourth step 308 of removing the dissolvable microneedle patch 100 from the skin. The removal of the dissolvable microneedle patch 100 detaches the base layer 102 after partial dissolution of the plurality of microneedles 104. In addition, the removal leaves no retained rigid microneedle structure within the intradermal tissue.

In an embodiment of the present disclosure, 5-90% of the solidified matrix dissolves prior to the removal of the dissolvable microneedle patch 100. The dissolution within the 5-90% range enables partial structural hydration before the detachment of the base layer 102. In another embodiment of the present disclosure, the solidified matrix may dissolve at a different percentage prior to the removal.

In an embodiment of the present disclosure, the non-encapsulated portion of the hormone releases upon the dissolution of the solidified matrix and the encapsulated portion releases upon degradation of the biodegradable polymer. The matrix dissolution produces initial hormone exposure. The biodegradable polymer degradation produces delayed hormone exposure. In another embodiment of the present disclosure, release ratio between the non-encapsulated portion and the encapsulated portion may vary. In an embodiment of the present disclosure, the hormone is selected from follicle stimulating hormone, luteinizing hormone, gonadotropin releasing hormone, progesterone, human chorionic gonadotropin, estrogen, or testosterone. In another embodiment of the present disclosure, the dissolvable microneedle patch 100 may deliver a different large-molecule hormone.

In an embodiment of the present disclosure, 10-100 IU of the hormone is administered to the subject. In another embodiment of the present disclosure, 40-60 IU of the hormone is administered to the subject. In yet another embodiment of the present disclosure, the dissolvable microneedle patch 100 may be used to administer a different International Unit dosage.

In an embodiment of the present disclosure, administration increases hormone concentration in the subject and the increased hormone concentration is maintained for 1-14 days. In another embodiment of the present disclosure, maintenance duration may differ according to therapeutic protocol. In an embodiment of the present disclosure, the dissolvable microneedle patch 100 is replaced daily over 2-14 days. In an embodiment of the present disclosure, the subject is a human. In an embodiment of the present disclosure, the human is female. In an embodiment of the present disclosure, the method includes administering at least one additional large molecule therapeutic by intradermal or subdermal injection.

The flowchart 300 includes applying the dissolvable microneedle patch 100, exerting sufficient force for the intradermal penetration, maintaining contact for the matrix dissolution, and removing the dissolvable microneedle patch 100 as core steps. The flowchart 300 may include additional clinical monitoring, concentration measurement, adjunct therapy, replacement scheduling, or protocol-driven adjustments. In an embodiment of the present disclosure, the method may include additional administration variations, dosage adjustments, hormone selections, dissolution percentages, release profiles, or replacement schedules.

Each microneedle of the plurality of microneedles 104 maintains mechanical integrity during penetration into intradermal tissue. Each microneedle of the plurality of microneedles 104 dissolves after the intradermal placement. The dissolution of the plurality of microneedles 104 releases a therapeutic payload without leaving a retained solid scaffold within the skin. The sustained release results from selection of material composition and incorporation strategy within the dissolving structural matrix. The sustained release does not depend on retention of non-dissolving microneedle hardware. In addition, the sustained release does not depend on hollow microneedles connected to external reservoirs. Moreover, the sustained release does not rely on a separable scaffold portion that remains embedded in tissue after patch removal.

Rapid-dissolving microneedle systems emphasize immediate exposure following dissolution. The rapid-dissolving systems couple structural dissolution with near-complete drug release over short durations. In contrast, the dissolvable microneedle patch 100 supports multi-day therapeutic availability and retains full dissolvability of the plurality of microneedles 104. The plurality of microneedles 104 do not persist as a rigid structural framework after administration. Further, non-dissolving or hybrid microneedle architectures achieve sustained delivery through persistent structural elements. Sustained release in those architectures results from retained hardware components. The dissolvable microneedle patch 100 achieves the sustained release through the controlled dissolution behavior of the structural matrix, which forms the plurality of microneedles 104. The structural matrix performs both the insertion and the release functions.

Additionally, implant-like microneedle systems deposit retained depots or detachable microneedle fragments within skin. The dissolvable microneedle patch 100 does not generate a retained implant or residual solid depot within intradermal tissue. Fertility treatment protocols require repeatable administration over defined short treatment windows. The dissolvable microneedle patch 100 supports repeated multi-day dosing cycles without residual material remaining between applications.

Further, the non-crosslinked structural matrix forms the primary structural material of plurality of microneedles 104. The non-crosslinked structural matrix functions as a load-bearing element during insertion into the intradermal tissue. In addition, the non-crosslinked structural matrix functions as the dissolution-controlled vehicle for the intradermal delivery of the large-molecule therapeutic. The non-crosslinked structural matrix supports delivery of fertility hormones in native molecular form. Chemically crosslinked hydrogel microneedle systems utilize covalent network formation to control mechanical properties and swelling behavior. The chemically crosslinked systems rely on photo crosslinked synthetic polymers or chemically modified biopolymers. The photo crosslinked polymer systems depend on ultraviolet-initiated reactions to form covalent bonds between the polymer chains. Chemically modified biopolymers incorporate reactive groups onto polymer backbones to enable network formation. In contrast, the plurality of microneedles 104 includes the non-crosslinked structural matrix that does not undergo covalent crosslinking. The non-crosslinked structural matrix does not require post-fabrication curing reactions. Further, the mechanical integrity of the plurality of microneedles 104 does not depend on crosslink density within a covalent hydrogel network. The non-crosslinked structural matrix dissolves through hydration-driven disruption of the polymer chain entanglement.

Particle-based sustained-release microneedle architectures embed drug-loaded microspheres or nanoparticles within a structural carrier. In those architectures, sustained release arises primarily from degradation kinetics of the embedded particles. In such systems, microneedles serve predominantly as insertion mechanisms. Release control depends largely on particle degradation rather than dissolution of the microneedle structural matrix. The dissolvable microneedle patch 100 employs the non-crosslinked structural matrix as the insertion structure and the dissolution-governing release framework. The release behavior depends on the controlled hydration and the dissolution of the plurality of microneedles 104.

Detachable or separable microneedle tip systems create a retained implant portion within skin. Detachable systems use effervescent layers or sacrificial interfaces to separate drug-containing tips. These architectures establish a solid depot retained within the intradermal tissue. Sustained release depends on persistence of the retained depot. The dissolvable microneedle patch 100 does not create a retained structural implant. The entire plurality of microneedles 104 dissolves after the insertion. Fertility treatment regimens require repeatable, protocol-driven dosing within defined short treatment windows. The non-crosslinked structural matrix supports repeat-use administration without accumulation of residual hardware.

Particle-based and detachable-tip systems may introduce variability in dose distribution, release kinetics, and tissue retention. The non-crosslinked structural matrix provides a chemically well-characterized and dissolution-controlled material platform. Furthermore, the chemically crosslinked systems introduce additional reactive constituents associated with crosslinking chemistry. The non-crosslinked structural matrix avoids introduction of crosslinking agents or polymer modification reagents. In addition, the non-crosslinked structural matrix integrates load-bearing insertion capability, controlled dissolution, and compatibility with fertility hormone delivery within a single unmodified polymer network.

Going further, the dissolvable microneedle patch 100 includes the large-molecule therapeutic within the plurality of microneedles 104 in more than one physical state. The plurality of microneedles 104 include the non-encapsulated fraction of the large-molecule therapeutic dispersed directly within the dissolving structural matrix. Also, the plurality of microneedles 104 include the encapsulated fraction of the large-molecule therapeutic contained within the biodegradable polymer carriers embedded within the dissolving structural matrix. The term “non-encapsulated fraction” refers to therapeutic molecules distributed directly throughout the polymer matrix without surrounding carrier material. The non-encapsulated fraction releases upon hydration-driven dissolution of the dissolving structural matrix. The term “encapsulated fraction” refers to therapeutic molecules contained within a biodegradable polymer carrier that forms a discrete physical domain within the dissolving structural matrix. The encapsulated fraction releases according to the degradation behavior of the biodegradable polymer carrier. The biodegradable polymer carrier degrades through hydrolytic or enzymatic mechanisms within the intradermal tissue. Degradation kinetics of the biodegradable polymer carrier differ from dissolution kinetics of the dissolving structural matrix. Upon application of the dissolvable microneedle patch 100, the plurality of microneedles 104 penetrate the intradermal tissue. The dissolving structural matrix hydrates and dissolves following the insertion. In addition, the dissolution of the dissolving structural matrix releases the non-encapsulated fraction of the large-molecule therapeutic into intradermal space. Moreover, the encapsulated fraction remains associated with the biodegradable polymer carrier until carrier degradation occurs. Further, the release of the encapsulated fraction occurs independently of the matrix dissolution once the biodegradable polymer carrier resides within the intradermal tissue. The dual-release mechanism separates the matrix-controlled release from the carrier-controlled release.

Single-mode dissolving microneedle systems incorporate therapeutic agents as uniform dispersions within fast-dissolving matrices. Release in single-mode systems depends primarily on dissolution rate of the structural matrix. These single-mode systems generate a rapid or bolus-type release profile. The dissolution behavior and the release kinetics remain tightly coupled within a single structural mechanism. Particle-only sustained-release architectures embed drug-loaded microspheres or nanoparticles as sole release-controlling components. In these architectures, the microneedles function primarily as insertion structures. Drug release in particle-only systems depends predominantly on the degradation kinetics of biodegradable particles. The matrix dissolution does not substantially contribute to temporal shaping of release.

The detachable or implant-like microneedle systems create a retained solid reservoir within the intradermal tissue. Those systems rely on persistence of the retained depot for extended release. The dissolvable microneedle patch 100 does not create a retained solid implant. The dual-release mechanism integrates two therapeutic populations within the dissolving plurality of microneedles 104. Fertility treatment regimens require protocol-driven timing characterized by an initial rise in the hormone concentration followed by maintenance within a defined therapeutic window. The non-encapsulated fraction enables an early-phase elevation in therapeutic exposure. In addition, the encapsulated fraction supports sustained maintenance of therapeutic concentration through biodegradable polymer carrier degradation. The dual-release mechanism enables the temporal structuring of the intradermal exposure. Moreover, the fertility treatment regimens involve repeated dosing over short treatment intervals. Reliance solely on retained depots or particulate systems may introduce variability in particle distribution and degradation behavior. The dual-release mechanism distributes the release control between the matrix dissolution and the carrier degradation. The separation of insertion mechanics and release modulation provides flexibility when adapting release profiles to fertility-specific dosing schedules. The dissolvable microneedle patch 100 integrates the non-encapsulated and the encapsulated therapeutic populations within the dissolving plurality of microneedles 104 to achieve the intradermal delivery governed by more than one release-controlling process.

The dissolvable microneedle patch 100 is configured to achieve the sustained intradermal release of the large-molecule therapeutic without chemical modification of the large-molecule therapeutic. The term “chemical modification” refers to covalent alteration of molecular structure of a therapeutic agent. The chemical modification includes conjugation, PEGylation, derivatization, prodrug formation, or attachment of functional groups to alter pharmacokinetics. The dissolvable microneedle patch 100 delivers the large-molecule therapeutic in native molecular form. The large-molecule therapeutic retains original chemical structure prior to incorporation into the plurality of microneedles 104. The release control arises from composition of the dissolving structural matrix and from encapsulation strategies integrated within the plurality of microneedles 104. The dissolving structural matrix regulates the hydration-driven dissolution kinetics. In addition, the biodegradable polymer carriers regulate the degradation-controlled release when the plurality of microneedles 104 include the encapsulated fraction of the large-molecule therapeutic. The sustained intradermal exposure results from physical formulation architecture rather than molecular alteration.

Extended-delivery peptide and protein systems often utilize covalent attachment of polyethylene glycol chains or other chemical substituents. Covalent conjugation strategies modify molecular weight and clearance behavior of therapeutic molecules. Some delivery systems employ prodrug strategies that convert an active molecule into a chemically altered precursor. Conversion back to the active molecule occurs after administration. The dissolvable microneedle patch 100 does not require the covalent conjugation or the prodrug transformation to achieve the multi-day release behavior. The large-molecule therapeutic remains chemically unaltered. In addition, certain sustained-release microneedle systems rely on long-acting injectable analogs adapted to microneedle formats. Long-acting analogs involve structural modification of native hormone molecules. The dissolvable microneedle patch 100 accommodates the fertility hormones without structural modification of the hormone molecule. The fertility hormones retain compatibility with established clinical formulations. Furthermore, some sustained-release systems rely on retained implants or depot structures that function independently of chemical modification of the drug. The dissolvable microneedle patch 100 does not depend on the retained solid implants to prolong exposure. The fertility treatment protocols rely on well-characterized, unmodified hormone formulations. Substitution with chemically modified analogs may require reformulation validation and compatibility assessment. The ability of the dissolvable microneedle patch 100 to deliver unmodified fertility hormones supports integration into protocol-driven dosing schedules. The sustained release results from the matrix composition, the dissolution behavior, and the optional encapsulation within the plurality of microneedles 104 rather than alteration of therapeutic chemistry. The dissolvable microneedle patch 100 achieves sustained intradermal hormone exposure through physical and structural design features and preserves the native molecular structure of the active therapeutic ingredient.

The present disclosure provides the dissolvable microneedle patch 100 in which geometry of the plurality of microneedles 104 is selected in conjunction with composition of the dissolving structural matrix. Each microneedle of plurality of microneedles 104 includes a height configured to penetrate epidermis and terminate within the intradermal tissue. The height lies within approximately 1000-3000 μm. Each microneedle of plurality of microneedles 104 includes a base dimension and a tip geometry configured to support insertion into skin without structural fracture. The tip geometry includes a tapered configuration that reduces insertion force. Array density of the plurality of microneedles 104 is selected to distribute externally applied force across the base layer 102 during administration. The array density reduces risk of collective buckling during the insertion. The term “co-optimization” refers to simultaneous selection of geometric parameters and polymer material parameters to achieve coordinated mechanical and dissolution performance. The geometric parameters may include height, base width, pitch, and tip configuration. The material parameters include the molecular weight of the non-crosslinked HMW hyaluronic acid, the viscosity of methylcellulose, total solids content, and presence of the non-crosslinked LMW hyaluronic acid.

Mechanical insertion reliability depends on interaction between the microneedle geometry and physical polymer network strength. Higher molecular weight polymer chains increase resistance to compressive deformation. In addition, the base width and the pitch selection influence load transfer during the insertion into the intradermal tissue. The increased base dimension enhances resistance to bending forces. Moreover, the dissolution timing depends on the polymer concentration and the microneedle volume. The geometry directly affects the hydration rate and the dissolution-controlled release kinetics. Further, incorporation of the large-molecule therapeutic within the plurality of microneedles 104 alters internal structural density. The geometry selection accounts for material strength reduction associated with therapeutic loading. Many microneedle systems optimize the geometry independently of the polymer composition. Some systems vary needle length or tip shape and maintain constant material formulation. Other systems modify the material composition without adjusting geometry to compensate for altered mechanical strength or dissolution properties.

For delivery of the large-molecule therapeutics, the insertion mechanics interact with payload incorporation and the polymer molecular weight. Assumptions derived from vaccine or small-molecule systems do not inherently address protein or hormone delivery constraints. The fertility hormones require reliable intradermal placement across repeated administrations. The geometry that lacks sufficient mechanical robustness may fracture during the insertion. Conversely, the geometry selected without regard to the dissolution kinetics may prolong matrix persistence or accelerate premature release. The coordination of the geometric and the material parameters reduces variability in delivered dose. The dissolvable microneedle patch 100 integrates the geometric design and the polymer composition as interdependent variables. The mechanical integrity during the insertion, the dissolution timing, and the intradermal delivery of the large-molecule therapeutics arise from the coordinated geometry-material design. The plurality of microneedles 104 reflects joint determination of the structural dimensions and the non-crosslinked polymer matrix properties to support reproducible intradermal fertility hormone delivery.

The plurality of microneedles 104 includes dimensional parameters selected within defined operational envelopes to maintain the insertion reliability and the dissolution behavior. Each microneedle of the plurality of microneedles 104 includes a height selected to enable penetration of the stratum corneum and epidermis and termination within the intradermal tissue. If the height of the plurality of microneedles 104 falls below an intradermal threshold, penetration depth decreases and a hormone deposits predominantly within superficial epidermal layers. Reduced penetration decreases exposure of intradermal vasculature and reduces systemic absorption efficiency. If height of the plurality of microneedles 104 exceeds an upper tolerance limit, externally required insertion force increases. The increased insertion force increases probability of user-dependent insertion variability. Moreover, excessive height increases likelihood of deeper-than-intended tissue penetration without proportional improvement in pharmacokinetic performance.

Each microneedle of the plurality of microneedles 104 includes the base width selected to resist compressive deformation during insertion. If the base width falls below a structural stability threshold, resistance to buckling decreases and probability of incomplete insertion increases. The incomplete insertion introduces variability in delivered dose across the plurality of microneedles 104. If the base width exceeds a practical upper bound, the insertion force increases due to increased cross-sectional resistance. Further, the increased base width reduces achievable packing density of the plurality of microneedles 104 across the base layer 102. The reduced packing density lowers cumulative payload capacity per the dissolvable microneedle patch 100.

Moreover, increased microneedle volume alters hydration dynamics by increasing diffusion path length for interstitial fluid penetration. Altered hydration dynamics modify dissolution timing and release kinetics. Accordingly, the geometric parameters of the plurality of microneedles 104 remain confined within a coordinated design envelope to preserve mechanical integrity during insertion, reproducible intradermal placement, and controlled dissolution-mediated delivery.

The dissolvable microneedle patch 100 includes the plurality of microneedles 104 arranged across the base layer 102 to distribute the hormone across multiple structural elements. The plurality of microneedles 104 increases cumulative microneedle volume available for incorporation of the hormone without requiring formation of a single large penetration structure. Each microneedle of the plurality of microneedles 104 contains a portion of a total therapeutic payload distributed across the array. The distribution of the hormone across the plurality of microneedles 104 reduces mechanical stress concentration that would otherwise occur in a single large needle geometry. In addition, the plurality of microneedles 104 enables incremental scaling of total payload through adjustment of microneedle count and preserve the insertion mechanics of the individual microneedles. Total deliverable dose per the dissolvable microneedle patch 100 depends on the microneedle height, the microneedle base width, the microneedle pitch, and the array density across the base layer 102. The array density defines cumulative microneedle volume within a defined patch footprint. Moreover, the microneedle density interacts with polymer concentration and the matrix strength to determine structural tolerance for hormone loading. Increasing the hormone concentration within the solidified matrix reduces effective mechanical modulus of each microneedle of the plurality of microneedles 104. The reduced mechanical modulus increases risk of bending or incomplete insertion under externally applied force. Accordingly, maximum hormone loading per microneedle reflects a balance between structural strength and payload mass. Further, distributing the hormone across the plurality of microneedles 104 reduces dose variability associated with incomplete insertion of any single microneedle. If a limited subset of microneedles exhibits reduced penetration depth, remaining microneedles maintain contribution to total delivered dose. The plurality of microneedles 104 provides redundancy that enhances dose reliability under real-world application variability. Moreover, the multi-needle architecture enables delivery of clinically relevant hormone quantities without requiring excessive enlargement of the individual microneedles. The enlargement of the individual microneedles would increase the insertion force and alter the dissolution kinetics. The plurality of microneedles 104 supports coordinated scaling of the payload capacity, the insertion reliability, and the dissolution behavior through distributed loading across the base layer 102.

Each microneedle of the plurality of microneedles 104 includes the solidified matrix formed from the non-crosslinked high molecular weight hyaluronic acid and the methylcellulose. The solidified matrix forms through solvent removal and physical polymer interaction without formation of the covalent crosslink junctions. The solidified matrix does not undergo chemical crosslinking reactions during fabrication. The solidified matrix does not undergo photo-initiated curing processes. The solidified matrix does not undergo ultraviolet curing, thermal curing, radical polymerization, or catalyst-driven network formation. Moreover, the polymer backbone of hyaluronic acid remains chemically unmodified. The polymer backbone of methylcellulose remains chemically unmodified. Structural integrity of each microneedle of the plurality of microneedles 104 results from physical chain entanglement and intermolecular hydrogen bonding between polymer chains. The hydrogen bonding interactions provide reversible cohesion under dry conditions. Upon exposure to an interstitial fluid, the hydration disrupts the hydrogen bonding interactions and permits dissolution of the solidified matrix. In addition, the absence of the covalent crosslink junctions allows the complete dissolution of each microneedle of the plurality of microneedles 104 following insertion. The dissolvable microneedle patch 100 avoids the residual crosslinked hydrogel fragments within the intradermal tissue.

Further, the absence of the chemical modification reactions eliminates introduction of crosslinking reagents, photoinitiators, residual monomers, or curing byproducts within the solidified matrix. The solidified matrix maintains a chemical composition limited to the hyaluronic acid, methylcellulose, the hormone, the optional biodegradable polymer carriers, and the optional pharmaceutically acceptable excipients. The physical-network architecture enables mechanical integrity during the insertion and preserves the complete dissolution behavior after the intradermal placement.

Further, the solidified matrix functions as a structural insertion element and as a dissolution-mediated delivery vehicle. The solidified matrix does not rely on permanently layered hydrogel architectures to regulate release behavior. The solidified matrix does not include a chemically crosslinked outer shell combined with a chemically distinct inner depot core. The solidified matrix does not include a retained structural scaffold that persists within the intradermal tissue after the dissolution of an outer layer. Moreover, the plurality of microneedles 104 does not employ a detachable tip configuration that separates from the base layer 102 to remain implanted within skin. The plurality of microneedles 104 does not rely on effervescent separation layers, sacrificial intermediate layers, or mechanically separable drug-containing segments. Further, the solidified matrix does not depend on a layered hydrogel system in which distinct polymer layers provide independently crosslinked release barriers. The release behavior of the hormone arises from the dissolution of the non-crosslinked matrix and, where present, the degradation of the biodegradable polymer carrier embedded within the matrix. The structural insertion function and the dissolution-mediated release function occur within the unified, fully dissolvable microneedle architecture. In addition, the absence of permanent layered hydrogel components eliminates the reliance on residual implant-like structures to sustain release. The complete dissolution of the plurality of microneedles 104 achieves delivery without the retained solid hydrogel segments or discrete depot layers formed by the crosslinked polymer domains. The dissolvable microneedle patch 100 maintains a structurally continuous, fully dissolvable microneedle architecture.

Further, the hormone is incorporated within the solidified matrix that maintains sufficient mechanical integrity during insertion into the intradermal tissue. The incorporation of the hormone within the solidified matrix alters physical properties of the matrix. The increasing hormone concentration within the solidified matrix reduces effective polymer volume fraction available for load-bearing function. The reduction of polymer volume fraction reduces compressive strength and flexural resistance of the plurality of microneedles 104. Moreover, excessive hormone loading disrupts the polymer chain entanglement density and reduces cohesive interaction between the hyaluronic acid and methylcellulose. TherReduced cohesive interaction lowers resistance to bending and buckling during insertion. Accordingly, the plurality of microneedles 104 includes a maximum hormone loading threshold defined by structural tolerance of the non-crosslinked matrix. Exceeding the structural tolerance threshold increases probability of incomplete penetration of skin. Incomplete penetration reduces reproducibility of delivered dose across plurality of microneedles 104. In addition, elevated hormone loading modifies the hydration kinetics following insertion. The increased hydrophilic solute fraction accelerates water ingress and may shorten dissolution time beyond a target window. The shortened dissolution time may shift release profile toward premature or less controlled exposure. Further, high loading may increase brittleness of a dried matrix during storage. The increased brittleness elevates probability of mechanical fracture during handling or application. The maximum deliverable payload per dissolvable microneedle patch 100 reflects the coordinated selection of microneedle geometry, the array density, the polymer concentration, and the hormone incorporation fraction. The plurality of microneedles 104 distributes total dose across multiple microneedles to remain within structural tolerance limits of individual microneedles. Accordingly, the dissolvable microneedle patch 100 achieves clinically relevant hormone delivery and preserves the insertion reliability and the dissolution-controlled release behavior.

Furthermore, the base layer 102 functions as a structural carrier for the plurality of microneedles 104 during handling and application. The base layer 102 maintains spatial alignment and perpendicular orientation of the plurality of microneedles 104 relative to a skin surface. In addition, the base layer 102 transmits externally applied force to the plurality of microneedles 104 during application. Moreover, the base layer 102 distributes externally applied force across a contact area to reduce localized overload on individual microneedles. The base layer 102 does not include mechanical penetration-limiting stops. The base layer 102 does not include depth-control projections or rigid insertion depth regulators. The penetration depth of the plurality of microneedles 104 depends on the microneedle height, the microneedle geometry, the mechanical properties of the solidified matrix, and a magnitude of externally applied force. In addition, the penetration depth depends on a mechanical resistance of the stratum corneum and the epidermal tissue. Accordingly, the base layer 102 operates as a passive structural support element. Further, the removal of the base layer 102 after onset of the dissolution does not disturb the intradermal placement because the plurality of microneedles 104 dissolve within the tissue. Accordingly, the dissolvable microneedle patch 100 achieves the intradermal delivery through the coordinated geometry and applied force.

Going further, upon insertion of the plurality of microneedles 104 into the intradermal tissue, the interstitial fluid contacts exposed surfaces of the solidified matrix. Initial hydration occurs through diffusion of the interstitial fluid into outer regions of the plurality of microneedles 104. The hydration increases mobility of the polymer chains within the solidified matrix. Moreover, the hydration weakens intermolecular hydrogen bonding interactions that maintain structural cohesion under dry conditions. Following the hydration, softening of the plurality of microneedles 104 occurs as the polymer chains transition from a rigid, glass-like state to a hydrated, viscoelastic state. The softening reduces mechanical stiffness of the solidified matrix. The progressive dissolution occurs as the hydrated polymer chains disengage and diffuse into the surrounding interstitial fluid. The dissolution rate depends on the molecular weight of the hyaluronic acid, the concentration of methylcellulose, the presence of the low molecular weight hyaluronic acid, the microneedle geometry, and local physiological conditions. In addition, the dissolution timing influences the release kinetics of the hormone dispersed within the solidified matrix. The release of the non-encapsulated fraction of the hormone begins during the hydration and accelerates during the dissolution. The plurality of microneedles 104 transitions through a defined sequence of the hydration, the softening, and the dissolution following the intradermal placement.

Following the dissolution of the plurality of microneedles 104, the hydrated polymer components remain within the intradermal tissue. The hydrated polymer components include the hyaluronic acid and methylcellulose in dissolved or partially dissolved form. The hydrated polymer components undergo physiological resorption through normal interstitial fluid turnover mechanisms. Moreover, degradation products of the biodegradable polymer carrier embedded within the solidified matrix undergo hydrolysis or enzymatic breakdown within the intradermal tissue. The degradation products enter systemic circulation or local metabolic pathways consistent with biocompatible material clearance. The dissolvable microneedle patch 100 does not leave behind rigid polymer fragments, non-dissolving scaffolds, detachable tips, or permanent hydrogel remnants within skin. The absence of retained rigid components reduces risk of chronic mechanical irritation. Accordingly, the plurality of microneedles 104 delivers the hormone while enabling the subsequent physiological clearance of the structural materials.

The incorporation of the hormone within the solidified matrix occurs under aqueous-compatible fabrication conditions. The fabrication process utilizes water as a primary solvent medium for the hyaluronic acid and methylcellulose. The fabrication process avoids use of harsh organic solvents that destabilize protein or peptide therapeutics. Moreover, the fabrication process avoids exposure of the hormone to extreme processing temperatures that induce denaturation or aggregation. The fabrication process avoids the chemical modification of the hormone through the covalent conjugation or the reactive crosslinking chemistries. Further, solvent removal during the matrix solidification occurs without radical polymerization or catalyst-driven curing reactions. The hydrophilic nature of the solidified matrix provides an aqueous microenvironment that maintains the structural integrity of the hormone during fabrication and storage. Accordingly, the dissolvable microneedle patch 100 supports the incorporation of native, unmodified large-molecule therapeutics without requiring the chemical alteration to achieve the sustained intradermal delivery.

In an embodiment of the present disclosure, the dissolvable microneedle patch 100 enables modulation of a therapeutic release profile through coordinated variation of the matrix composition and the encapsulation ratio within the plurality of microneedles 104. The term “release profile” refers to a temporal pattern of therapeutic availability within the intradermal tissue following insertion of the plurality of microneedles 104. The term “encapsulation ratio” refers to a relative proportion between the non-encapsulated fraction of the large-molecule therapeutics and the encapsulated fraction of the large-molecule therapeutic within the solidified matrix. In an embodiment of the present disclosure, the modulation of the release profile occurs through adjustment of the molecular weight of the non-crosslinked high molecular weight hyaluronic acid. In another embodiment of the present disclosure, the modulation of the release profile occurs through adjustment of the relative concentration of the non-crosslinked high molecular weight hyaluronic acid within the solidified matrix. In yet another embodiment of the present disclosure, the modulation of the release profile occurs through the adjustment of the concentration of methylcellulose within the solidified matrix.

If a proportion of the non-encapsulated fraction increases, an early-phase exposure following hydration of the plurality of microneedles 104 increases. If a proportion of the encapsulated fraction increases, a sustained-phase exposure governed by the degradation of the biodegradable polymer carrier increases. Moreover, increase in the molecular weight of the non-crosslinked high molecular weight hyaluronic acid reduces a dissolution rate of the solidified matrix. In addition, increase in the concentration of methylcellulose increases mechanical density of the solidified matrix and modifies hydration kinetics. Further, inclusion of the non-crosslinked low molecular weight hyaluronic acid increases the dissolution modulation through alteration of the polymer chain entanglement density. Adjustment of the encapsulation ratio separates matrix-controlled release from carrier-controlled release. The separation between the dissolution-driven release and the degradation-driven release enables construction of the multi-phase release profile.

In an embodiment of the present disclosure, the plurality of microneedles 104 provide the immediate release profile. In another embodiment of the present disclosure, the plurality of microneedles 104 provide the sustained release profile. In yet another embodiment of the present disclosure, the plurality of microneedles 104 provide a multi-phase release profile that includes an initial dissolution-driven phase and a subsequent degradation-driven phase. In yet another embodiment of the present disclosure, the plurality of microneedles 104 provide a controlled release profile tailored to a protocol-driven fertility treatment schedule. In yet another embodiment of the present disclosure, the solidified matrix and the encapsulation ratio may define a different therapeutic release profile. In an embodiment of the present disclosure, the dissolvable microneedle patch 100 supports the intradermal delivery of the large-molecule therapeutic selected from a class of fertility-related hormones and biologics. The term “fertility hormone” refers to a hormonally active polypeptide or protein administered as part of an assisted reproduction protocol. In an embodiment of the present disclosure, the large-molecule therapeutic includes a gonadotropin. In another embodiment of the present disclosure, the large-molecule therapeutic includes follicle-stimulating hormone. In yet another embodiment of the present disclosure, the large-molecule therapeutic includes luteinizing hormone. In yet another embodiment of the present disclosure, the large-molecule therapeutic includes human chorionic gonadotropin. In yet another embodiment of the present disclosure, the large-molecule therapeutic includes a gonadotropin-releasing hormone agonist. In yet another embodiment of the present disclosure, the large-molecule therapeutic includes a gonadotropin-releasing hormone antagonist. In yet another embodiment of the present disclosure, the large-molecule therapeutic includes progesterone. In yet another embodiment of the present disclosure, the large-molecule therapeutic includes a recombinant fertility hormone. In yet another embodiment of the present disclosure, the large-molecule therapeutic includes a urinary-derived fertility hormone preparation.

The term “recombinant fertility hormone” refers to a hormone produced through recombinant DNA expression within a host cell system. The term “urinary-derived fertility hormone preparation” refers to a hormone isolated from biological fluids and purified for therapeutic administration. In an embodiment of the present disclosure, the plurality of microneedles 104 deliver the fertility hormone having a molecular weight within a peptide range. In another embodiment of the present disclosure, the plurality of microneedles 104 deliver the fertility hormone having a molecular weight within a glycoprotein range. In yet another embodiment of the present disclosure, the plurality of microneedles 104 deliver a fertility hormone having a multi-subunit structure. In addition, the dissolvable microneedle patch 100 accommodates the fertility hormones that exhibit different hydrodynamic radii and tertiary conformations. Moreover, the dissolvable microneedle patch 100 accommodates the fertility hormones that exhibit different aqueous solubility profiles. Further, the dissolvable microneedle patch 100 accommodates the fertility hormones that exhibit different stability characteristics under physiological conditions. In an embodiment of the present disclosure, the plurality of microneedles 104 may deliver the large-molecule therapeutic other than the fertility hormone. In another embodiment of the present disclosure, the plurality of microneedles 104 deliver a peptide therapeutic used in endocrine modulation. In yet another embodiment of the present disclosure, the plurality of microneedles 104 deliver an antibody fragment. In yet another embodiment of the present disclosure, the plurality of microneedles 104 deliver a nucleic acid construct intended for localized intradermal action. In addition, the dissolvable microneedle patch 100 supports delivery of the large-molecule therapeutics that require repeated administration within a defined treatment cycle.

Moreover, the dissolvable microneedle patch 100 supports delivery of the large-molecule therapeutics that exhibit batch-to-batch structural consistency across different active ingredients within the disclosed class. In yet another embodiment of the present disclosure, the plurality of microneedles 104 deliver a combination of the fertility hormones administered sequentially or concurrently within a treatment cycle. In yet another embodiment of the present disclosure, the plurality of microneedles 104 deliver the large-molecule therapeutic selected from a different subclass of protein or peptide agents compatible with the intradermal administration. In yet another embodiment of the present disclosure, the plurality of microneedles 104 may deliver a different large-molecule therapeutic within a broader biologic class.

In an embodiment of the present disclosure, the dissolvable microneedle patch 100 supports administration within the fertility treatment protocol that requires repeated dosing over a defined treatment interval. The term “fertility treatment protocol” refers to a clinically structured dosing schedule that governs timing, quantity, and sequence of hormone administration during assisted reproductive procedures. In an embodiment of the present disclosure, the fertility treatment protocol includes daily administration over a stimulation phase. In another embodiment of the present disclosure, the fertility treatment protocol includes administration at multi-day intervals. In yet another embodiment of the present disclosure, the fertility treatment protocol includes a controlled ovarian stimulation cycle. In yet another embodiment of the present disclosure, the fertility treatment protocol includes a luteal phase support cycle. The dissolvable microneedle patch 100 includes the plurality of microneedles 104 that dissolve completely after the intradermal placement. The complete dissolution eliminates retention of rigid structural hardware within the intradermal tissue between successive applications. In addition, the complete dissolution permits reapplication at the same anatomical region without interference from residual solid elements. Moreover, absence of retained scaffold segments prevents accumulation of persistent structural fragments during multi-dose treatment cycles.

In an embodiment of the present disclosure, the dissolvable microneedle patch 100 supports repeat placement on adjacent skin regions within a treatment area. In another embodiment of the present disclosure, the dissolvable microneedle patch 100 supports rotation of placement sites during a fertility cycle. In yet another embodiment of the present disclosure, the dissolvable microneedle patch 100 supports sequential administration aligned with protocol-defined monitoring milestones. The term “protocol-defined monitoring milestone” refers to a clinically determined time point at which hormone levels or follicular development undergo assessment. In addition, the dissolvable microneedle patch 100 supports repeat administration without introduction of surgically implanted depots. Moreover, the dissolvable microneedle patch 100 supports repeat administration without requirement for removal of retained microneedle remnants. Further, repeat application does not require extraction of residual polymer components from prior dosing events. In an embodiment of the present disclosure, the plurality of microneedles 104 provide hormone exposure within a defined therapeutic window that concludes prior to a subsequent scheduled administration. In another embodiment of the present disclosure, the plurality of microneedles 104 provide hormone exposure that integrates within a controlled ovarian stimulation timeline. In yet another embodiment of the present disclosure, the plurality of microneedles 104 provide hormone exposure that supports synchronization of follicular maturation.

In addition, the dissolvable microneedle patch 100 enables the user-administered application within a home-care setting consistent with fertility treatment regimens. Moreover, the dissolvable microneedle patch 100 reduces reliance on repeated hypodermic injection events during a stimulation cycle. Further, elimination of injection hardware between administrations supports improved protocol adherence during repeated dosing schedules. In yet another embodiment of the present disclosure, the dissolvable microneedle patch 100 supports repeated administration within a different hormone-driven reproductive protocol. In yet another embodiment of the present disclosure, the dissolvable microneedle patch 100 may support repeated administration within an alternate cyclic endocrine therapy schedule.

In an embodiment of the present disclosure, the dissolvable microneedle patch 100 supports scalable manufacturing within disclosed material composition ranges and geometric parameter ranges. The term “scalable manufacturing” refers to reproducible fabrication of the dissolvable microneedle patch 100 across different batch sizes and maintaining dimensional fidelity and compositional uniformity. In an embodiment of the present disclosure, the plurality of microneedles 104 are fabricated in arrays that maintain consistent microneedle height, consistent microneedle base width and consistent microneedle pitch across a production batch. The dimensional consistency across the plurality of microneedles 104 preserves the insertion reliability across manufactured units. In addition, the maintenance of uniform total polymer solids content across production batches preserves the dissolution timing characteristics.

Moreover, the maintenance of the uniform relative concentrations of the non-crosslinked high molecular weight hyaluronic acid, methylcellulose, and optional non-crosslinked low molecular weight hyaluronic acid preserves the mechanical strength consistency across manufactured units. In an embodiment of the present disclosure, the dissolvable microneedle patch 100 is produced using mold-based replication that ensures geometric repeatability of the plurality of microneedles 104. In another embodiment of the present disclosure, the dissolvable microneedle patch 100 is produced using micro-fabrication tooling that maintains micron-scale dimensional tolerances. In yet another embodiment of the present disclosure, the dissolvable microneedle patch 100 is produced in multi-cavity mold configurations that permit parallel production of multiple patch units. The term “micron-scale dimensional tolerance” refers to dimensional variation maintained within a range that does not materially affect insertion depth or dissolution behavior. In addition, the polymer concentration control during fabrication preserves batch-to-batch structural consistency. Moreover, viscosity control of methylcellulose during the formulation preparation preserves mold-filling uniformity across production cycles. Further, control of the encapsulation ratio within the plurality of microneedles 104 preserves the release profile reproducibility across manufactured lots.

In an embodiment of the present disclosure, the dissolvable microneedle patch 100 undergoes dimensional verification to confirm microneedle height uniformity. In an embodiment of the present disclosure, the dissolvable microneedle patch 100 undergoes mechanical strength testing to confirm insertion resistance parameters. In an embodiment of the present disclosure, the dissolvable microneedle patch 100 undergoes dissolution profiling to confirm release window conformity. Quality control evaluation preserves functional equivalence across scaling transitions from pilot batches to full-scale production. In addition, the material selection limited to the non-crosslinked polymers reduces variability introduced by crosslink density fluctuations that occur in chemically cured hydrogel systems. Moreover, absence of the photoinitiated curing steps removes ultraviolet intensity variability from large-scale production environments. Further, the dissolvable microneedle patch 100 supports scaling without incorporation of detachable microneedle segments that require secondary assembly operations. In an embodiment of the present disclosure, the manufacturing scale increases with maintenance of the geometric-material coordination within previously disclosed parameter ranges.

The present disclosure provide numerous technical advantages. The dissolvable microneedle patch 100 enables the reliable intradermal delivery of at least one large molecule therapeutic without retention of the structural elements within the tissue. The plurality of microneedles 104 maintain the mechanical rigidity sufficient to penetrate the stratum corneum and the epidermis and terminate within the intradermal tissue. The mechanical rigidity arises from the polymer chain entanglement and the intermolecular hydrogen bonding within the non-crosslinked matrix. In addition, the plurality of microneedles 104 undergo progressive hydration following insertion into the intradermal tissue. Hydration disrupts reversible intermolecular interactions within the matrix. The disruption of the intermolecular interactions initiates the controlled dissolution of the solidified matrix. The complete dissolution of the plurality of microneedles 104 occurs within the physiological tissue without leaving behind rigid fragments. Moreover, the dissolvable microneedle patch 100 excludes non-dissolving microneedles, detachable microneedle tips, hollow delivery conduits, and retained polymer scaffolds. The absence of retained structural components prevents residual hardware accumulation within the tissue following administration. Further, the dissolution-mediated intradermal deposition distributes therapeutic material directly within the interstitial fluid present in the dermal tissue. The intradermal placement within vascularized dermis supports systemic uptake of the large molecule therapeutic. Also, removal of the base layer 102 following completion of the administration leaves no implantable structure within the tissue. Accordingly, the dissolvable microneedle patch 100 enables minimally invasive intradermal administration without reliance on retained implantable delivery systems.

Further, the dissolvable microneedle patch 100 provides the sustained intradermal delivery of the at least one large molecule therapeutic over multiple days despite the full dissolution of the plurality of microneedles 104. The sustained exposure results from the coordinated interaction between the matrix composition and the fractional encapsulation of the therapeutic within at least one biodegradable polymer dispersed in the matrix. The matrix composition regulates the hydration rate and the dissolution kinetics following insertion into the intradermal tissue. The non-encapsulated fraction becomes available following the hydration-mediated matrix erosion, and the encapsulated fraction remains physically confined within the biodegradable polymer carriers positioned in the intradermal tissue after the matrix dissolution. The biodegradable polymer undergoes gradual degradation under physiological conditions over a defined duration ranging between approximately 1-14 days. This extends the therapeutic availability beyond the structural dissolution of the plurality of microneedles 104. Moreover, the separation between the matrix dissolution behavior and the biodegradable polymer degradation behavior establishes temporal decoupling between insertion mechanics and extended drug release. The degradation of the biodegradable polymer regulates continued exposure within the intradermal tissue. Accordingly, the cumulative release profile maintains therapeutic levels over multiple days without reliance on retained rigid structures, detachable implants, or non-dissolving scaffolds. Further, the sustained delivery occurs using the hydrophilic, non-crosslinked polymers that fully dissolve within the tissue and preserve the structural integrity during the insertion. The dissolvable microneedle patch 100 enables multi-day intradermal exposure using a structure that completely dissolves after administration.

Furthermore, the dissolvable microneedle patch 100 enables the controlled intradermal delivery of the at least one large molecule therapeutic in the native molecular form without chemical modification of the therapeutic agent. The therapeutic is physically dispersed within the solidified matrix and partially encapsulated within the at least one biodegradable polymer without covalent attachment, conjugation, PEGylation, or prodrug conversion. Physical entrapment within the matrix stabilizes the therapeutic during fabrication and storage. The encapsulation within the biodegradable polymer provides delayed release through the polymer degradation kinetics. The release mechanism depends on matrix erosion and biodegradable carrier degradation. Moreover, preservation of the native molecular structure avoids changes in receptor binding affinity, immunogenic profile, or pharmacodynamic behavior. The dissolvable microneedle patch 100 supports administration of clinically established hormone formulations and biologic preparations without reformulation into chemically altered derivatives. Maintenance of structural integrity of the therapeutic ensures compatibility with existing fertility treatment protocols and dosing regimens. Further, elimination of chemical modification steps reduces manufacturing complexity and avoids introduction of reactive crosslinking agents or synthetic coupling chemistries that may compromise molecular stability. The therapeutic remains chemically identical before incorporation into the plurality of microneedles 104 and after release into intradermal tissue. The delivery platform therefore operates as a physical carrier system rather than a chemically transformative system.

Also, the dissolvable microneedle patch 100 improves dose reliability for protocol-driven fertility treatment regimens requiring repeated and controlled administration of the at least one hormone. The plurality of microneedles 104 are arranged across the base layer 102 in a predefined geometric array with defined microneedle height, base width, and pitch parameters that collectively promote uniform insertion into the intradermal tissue. Uniform dimensional characteristics across the plurality of microneedles 104 reduce insertion variability and support consistent intradermal placement of the hormone at multiple distributed sites within an administration area. In addition, the plurality of microneedles 104 contain an equal or approximately equal quantity of the hormone in certain embodiments. This enables distribution of the total administered dose across multiple structurally consistent penetration elements. The distributed deposition reduces dependence on a single delivery site and mitigates variability associated with the incomplete insertion of isolated microneedles. Mechanical stability derived from the non-crosslinked polymer matrix supports penetration without bending or fracture, which enhances delivery consistency across the microneedle array. Moreover, the dissolvable microneedle patch 100 excludes retained depots, detachable microneedle segments, and non-dissolving scaffold components that remain embedded within tissue between administrations.

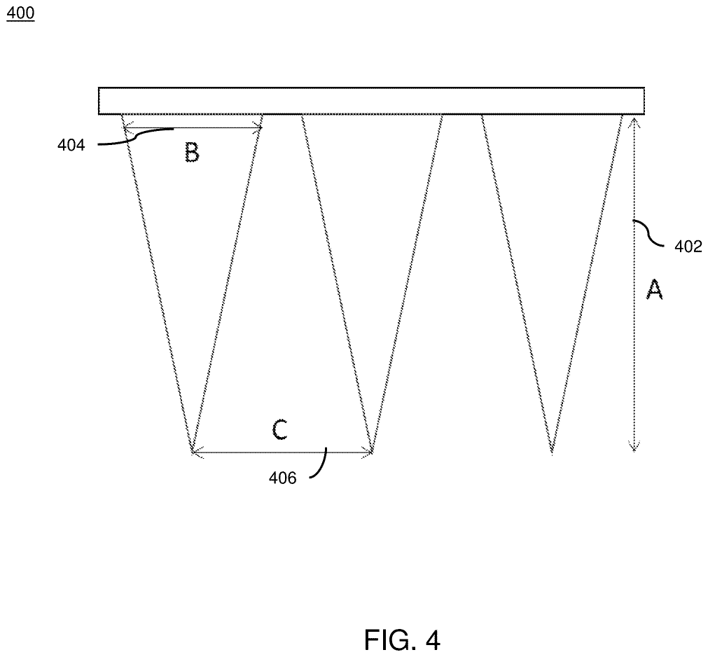

Further, the therapeutic release behavior does not rely exclusively on the same parameters that determine insertion strength. The release kinetics depend upon the hydration-mediated matrix dissolution and the degradation behavior of the at least one biodegradable polymer encapsulating a portion of the therapeutic. Adjustment of the encapsulation fraction, the biodegradable polymer composition, and the polymer molecular weight modifies release duration without necessarily altering geometric parameters governing insertion. Accordingly, the plurality of microneedles 104 retain mechanical robustness during administration even when therapeutic exposure duration is adjusted to accommodate different treatment requirements. Further, elimination of complete dependence on matrix density for release modulation avoids trade-offs that otherwise arise in systems where increased mechanical strength directly reduces dissolution rate. The dissolvable microneedle patch 100 maintains consistent insertion performance and permits flexible tuning of the therapeutic kinetics through material composition and encapsulation strategy. Further, the dissolvable microneedle patch 100 is suitable for repeated administration within a single fertility treatment cycle requiring multiple hormone dosing events over a defined interval. FIG. 4 illustrates a schematic representation of dimensional parameters of the plurality of microneedles in accordance with various embodiments of the present disclosure. The dimensional characteristics of the plurality of microneedles 104 are defined using geometrical parameters that govern penetration performance, mechanical stability, and spatial distribution of the microneedle array extending from the base layer 102. Each microneedle of the plurality of microneedles 104 extends outward from the base layer 102 substantially perpendicular to a surface of the base layer 102. The dimensional parameters include a microneedle height 402, a microneedle base width 404, and a microneedle pitch 406 between adjacent microneedles.

The microneedle height 402 represents a longitudinal dimension measured from an interface between the plurality of microneedles 104 and the base layer 102 to a distal tip of each microneedle. In certain embodiments, the microneedle height 402 ranges between approximately 1000 μm and 3000 μm. Selection of the microneedle height 402 enables penetration through the stratum corneum and epidermal layers while terminating within intradermal tissue regions suitable for therapeutic administration. The selected height supports formation of microscale transport pathways across the skin barrier while avoiding penetration into deeper nerve-rich regions associated with pain sensation.

In certain embodiments, the microneedle height 402 is selected such that individual microneedles terminate within an epidermal layer or within a dermal layer following insertion into skin. Selection of penetration depth depends upon a desired administration profile while maintaining minimally invasive interaction with tissue.

In certain embodiments, substantially uniform microneedle height 402 across the plurality of microneedles 104 promotes consistent insertion depth during application of the dissolvable microneedle patch 100. Uniform dimensional characteristics reduce variability in intradermal engagement across the microneedle array. In alternative embodiments, microneedles having differing heights may be distributed across the base layer 102 to accommodate variation in skin compliance across an administration site.

The microneedle base width 404 corresponds to a transverse dimension measured across a proximal region of each microneedle adjacent to the base layer 102. The microneedle base width 404 ranges between approximately 100 μm and 500 μm. The base dimension provides structural reinforcement required to withstand compressive forces applied during insertion. Increased base dimension enhances resistance to bending, buckling, or fracture of the plurality of microneedles 104 during manual application.

The dimensional relationship between the microneedle height 402 and the microneedle base width 404 establishes an aspect ratio selected to balance penetration efficiency with structural robustness. The defined aspect ratio enables efficient transfer of externally applied force toward distal microneedle tips while maintaining sufficient mechanical support near the base layer 102.

Each microneedle includes a tapered structure extending between the microneedle base width 404 and the distal tip. Progressive reduction in cross-sectional area concentrates insertion force at the tip region, enabling penetration of the outer skin barrier using practical manual application forces.

The microneedle pitch 406 represents spacing between adjacent microneedles within the plurality of microneedles 104. In certain embodiments, the microneedle pitch 406 ranges between approximately 100 μm and 3000 μm. Selection of the microneedle pitch 406 enables mechanical independence of individual microneedles during insertion and reduces interaction between neighboring microneedles under applied force.

Appropriate selection of the microneedle pitch 406 supports uniform distribution of insertion forces across the dissolvable microneedle patch 100 and minimizes localized stress accumulation. Controlled spacing reduces risk of collective deformation and promotes engagement of substantially all microneedles during application.

Dimensional parameters illustrated in FIG. 4 collectively influence overall microneedle density across the base layer 102. In certain embodiments, the dissolvable microneedle patch 100 includes approximately 10 to 1000 microneedles, and in certain embodiments 50 to 1000 microneedles, arranged according to the microneedle pitch 406 across a defined patch surface area.

The plurality of microneedles 104 may be of various shapes and sizes. In some embodiments, the plurality of microneedles 104 include a tapered tip that extends away from the base layer, optionally the tip having a contact area less than that of a 30-gauge hypodermic needle. In some embodiments, the plurality of microneedles 104 include microneedles of equal lengths or approximately equal lengths. In some embodiments, the plurality of microneedles include microneedles of at least two different lengths. In some embodiments, the plurality of microneedles has a length to terminate in the dermis. In some embodiments, the plurality of microneedles has a length to terminate in the epidermis. In some embodiments, each microneedle in the plurality of microneedles 104 has a length of 1-3mm. In certain embodiments, microneedle geometry associated with the dimensional parameters includes conical or pyramidal configurations including square pyramidal, trigonal pyramidal, hexagonal pyramidal, heptagonal pyramidal, octagonal pyramidal, nonagonal pyramidal, or decagonal structures. Experimental observations indicates that microneedle geometry and dimensional proportions influence penetrability, flexibility, and structural strength during insertion. Square-shaped microneedles may provide improved mechanical performance relative to conical geometries under selected dimensional conditions.

The dimensional configuration defined by the microneedle height 402, the microneedle base width 404, and the microneedle pitch 406 supports reproducible manufacturing using microneedle molds. Controlled dimensions enable uniform mold filling, reduction of air entrapment, and consistent formation of dissolvable microneedle arrays during air-drying or freeze-drying fabrication processes.

In further embodiments, dimensional characteristics of the plurality of microneedles 104 include structural features associated with microneedle tip geometry and penetration depth selection. Each microneedle includes a distal tip region having a contact area smaller than a contact area of a 30-gauge hypodermic needle. Reduction of tip contact area concentrates application force during administration and facilitates penetration through the stratum corneum using manual application pressure.

The plurality of microneedles 104 include microneedles having substantially equal lengths across the dissolvable microneedle patch 100 to promote uniform insertion performance. In alternative embodiments, the plurality of microneedles 104 include microneedles having at least two different lengths arranged across the base layer 102 to accommodate variations in skin compliance or application conditions.

In certain embodiments, each microneedle includes a tip portion representing approximately 5% to 95% of a total microneedle volume. Variation in relative tip volume enables adjustment of penetration behavior and structural strength while maintaining compatibility with dissolvable microneedle construction.

In certain embodiments, the plurality of microneedles 104 are boreless structures formed as solid dissolvable elements. In alternative embodiments, individual microneedles include a bore extending along at least a portion of a microneedle length, the bore having a gauge ranging between approximately 18 gauge and 32 gauge.

Dimensional parameters associated with the microneedle height 402, the microneedle base width 404, the microneedle pitch 406, and microneedle tip configuration collectively influence penetration strength of the plurality of microneedles 104. Mechanical strength sufficient for skin penetration is achieved through interaction between microneedle geometry and matrix material properties, thereby enabling resistance to crushing or deformation during application while maintaining dissolvability following administration.

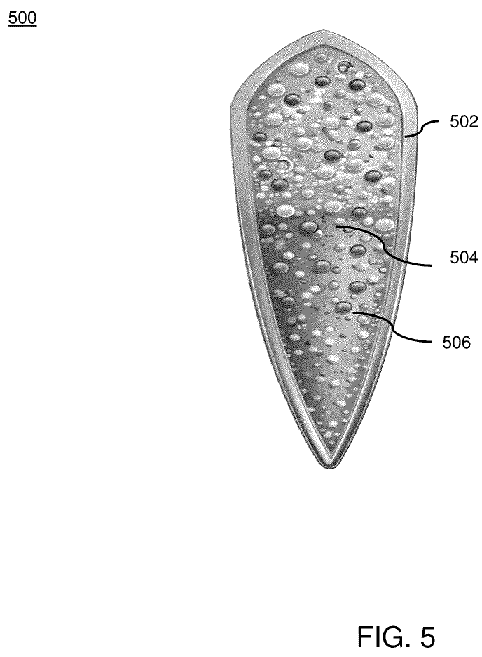

FIG. 5 illustrates a schematic cross-sectional representation 500 of therapeutic distribution within an individual microneedle of the plurality of microneedles 104 of the dissolvable microneedle patch 100, in accordance with various embodiments of the present disclosure. FIG. 5 illustrates structural organization of at least one large molecule therapeutic within a solidified matrix forming each microneedle of the plurality of microneedles 104. The illustrated configuration represents incorporation of the therapeutic in multiple physical states within a single dissolvable microneedle structure to enable controlled intradermal administration following insertion into skin.

Each microneedle of the plurality of microneedles 104 includes a solid matrix formed from non-crosslinked hyaluronic acid and methylcellulose. The matrix functions as a structural body supporting mechanical penetration while simultaneously serving as a carrier medium for therapeutic incorporation. The matrix includes regions containing therapeutic material embedded within the microneedle volume.

An embedded therapeutic region 502 represents a portion of at least one large molecule therapeutic incorporated directly within the matrix forming the microneedle structure. The embedded therapeutic region 502 includes therapeutic molecules positioned throughout the internal volume of the microneedle during formation of the solidified matrix.

In certain embodiments, the embedded therapeutic region 502 includes hormone molecules incorporated during preparation of a matrix material prior to microneedle molding. Incorporation within the matrix establishes physical immobilization of the therapeutic without covalent modification of molecular structure.

Placement of the therapeutic within the embedded therapeutic region 502 enables distribution of therapeutically active material throughout the microneedle body. Placement of the therapeutic within the embedded therapeutic region 502 maintains compatibility with biologically sensitive macromolecules. The macromolecules include proteins, peptides, and reproductive hormones. The hydrophilic polymer environment formed by hyaluronic acid supports stabilization of large molecule therapeutics during fabrication and storage.

A dispersed therapeutic region 504 represents a non-encapsulated fraction of the at least one large molecule therapeutic distributed within the matrix material as discrete molecular or particulate domains.

The dispersed therapeutic region 504 includes therapeutic material distributed substantially throughout the dissolvable matrix without confinement inside carrier structures. Distribution within the dispersed therapeutic region 504 results from mixing of therapeutic material within the matrix precursor prior to casting into microneedle molds.

Presence of the dispersed therapeutic region 504 enables immediate availability of therapeutic material following exposure of the microneedle matrix to interstitial fluid after insertion into intradermal tissue. Hydration of the matrix initiates dissolution of surrounding polymer material, permitting release of the dispersed therapeutic fraction without dependence upon degradation of secondary carrier materials.

Distribution of therapeutic material within the dispersed therapeutic region 504 supports rapid establishment of therapeutic exposure following administration. The distribution maintains the structural integrity of the microneedle during insertion.

An encapsulated therapeutic region 506 includes a portion of the at least one large molecule therapeutic contained within biodegradable polymer carriers dispersed inside the microneedle matrix. The encapsulated therapeutic region 506 includes therapeutic material enclosed within biodegradable polymers including but not limited to polyethylene glycol (PEG), polyvinyl alcohol (PVA), poly(lactic-co-glycolic acid) (PLGA), polycaprolactone (PCL), poly(lactic acid) (PLA), polyanhydrides, polyurethanes, polysaccharides, proteins, or polynucleotides. Encapsulation physically separates a portion of the therapeutic from direct interaction with surrounding matrix material. Encapsulation protects structurally sensitive large molecule therapeutics from environmental exposure during fabrication and administration while enabling delayed release governed by degradation behavior of the biodegradable polymer.

Following dissolution of the surrounding microneedle matrix after insertion into skin, biodegradable carriers located within the encapsulated therapeutic region 506 remain within intradermal tissue and gradually degrade over a controlled period ranging between approximately 1-14 days. Degradation of the biodegradable polymer releases the encapsulated therapeutic fraction over an extended duration.

Further, FIG. 5 illustrates coexistence of a non-encapsulated therapeutic population located within the embedded therapeutic region 502 and dispersed therapeutic region 504 and an encapsulated therapeutic population located within the encapsulated therapeutic region 506. In an embodiment, less than 100% of the therapeutic is encapsulated within biodegradable carriers. Presence of both therapeutic populations establishes separation between structural dissolution of the microneedle and subsequent therapeutic release behavior. The illustrated configuration enables administration of large molecule therapeutics without reliance on retained non-dissolving microneedle structures or implantable depots. The microneedle matrix dissolves following administration while biodegradable carriers independently regulate continued therapeutic availability within tissue.

Structural arrangement illustrated in FIG. 5 supports incorporation of large molecule therapeutics including peptide hormones, protein hormones, biologics, and reproductive hormones without chemical modification of the therapeutic agent.

The aqueous-compatible polymer matrix avoids exposure of the therapeutic to elevated temperature, organic solvents, or chemical crosslinking reactions. Physical incorporation within the matrix preserves biological activity of large molecule therapeutics during manufacturing and administration.

Distribution of therapeutic material across the embedded therapeutic region 502, dispersed therapeutic region 504, and encapsulated therapeutic region 506 enables controlled intradermal administration characterized by initial availability of non-encapsulated therapeutic following matrix hydration and prolonged availability governed by degradation of biodegradable carriers. The structural configuration enables delivery of therapeutically effective quantities of large molecule therapeutics while maintaining full dissolvability of the plurality of microneedles 104 following insertion.

Following insertion of the plurality of microneedles 104 into skin, the structural configuration illustrated in FIG. 5 enables controlled interaction between the microneedle matrix and physiological fluid present within intradermal tissue.

Upon penetration of the skin barrier, interstitial fluid enters external surfaces of each microneedle. Contact between bodily fluid and the matrix initiates hydration of the non-crosslinked hyaluronic acid and methylcellulose forming the microneedle structure. Hydration progressively reduces mechanical rigidity of the matrix and maintains positional stability of inserted microneedles during an initial administration period. In certain embodiments, dissolution rate of the plurality of microneedles is controlled through selection of polymer molecular weight, polymer concentration within the matrix material, and geometrical parameters of the microneedles including height, base dimension, and aspect ratio.

Dissolution of the plurality of microneedles 104 proceeds from outer surface regions toward interior regions of the microneedle body. Progressive dissolution releases therapeutic material located within the embedded therapeutic region 502 and the dispersed therapeutic region 504 directly into surrounding intradermal tissue.

Release of therapeutic material from the embedded therapeutic region 502 occurs as dissolution exposes previously immobilized therapeutic molecules to hydrated tissue environments. Release from the dispersed therapeutic region 504 occurs substantially concurrently with matrix erosion due to direct exposure of non-encapsulated therapeutic material to interstitial fluid.

In certain embodiments, dissolution of the plurality of microneedles 104 occurs over a period ranging between approximately 1 day and 14 days following administration. Dissolution time depends upon matrix composition, microneedle dimensions, hydration rate, and local physiological conditions within the skin.

During dissolution, structural material forming the microneedles converts into hydrated biocompatible polymer components that undergo physiological resorption or clearance through normal biological pathways. The dissolvable construction prevents retention of rigid microneedle fragments or sharp waste within tissue following administration.

After partial or complete dissolution of the microneedle matrix, biodegradable carriers associated with the encapsulated therapeutic region 506 remain positioned within intradermal tissue.

The biodegradable polymer surrounding the encapsulated therapeutic undergoes gradual degradation following exposure to physiological conditions. Degradation occurs through hydrolytic or diffusion-mediated mechanisms depending upon polymer selection. Degradation of the biodegradable polymer releases the encapsulated portion of the therapeutic over a controlled duration ranging between approximately 1 day and 14 days after insertion. Release from the encapsulated therapeutic region 506 continues after structural dissolution of the plurality of microneedles 104. Separation between the matrix dissolution and the biodegradable carrier degradation enables sustained intradermal exposure without dependence upon retained non-dissolving microneedles, detachable tips, or implantable delivery structures.

The therapeutic distribution within an individual microneedle establishes sequential therapeutic availability. The sequential therapeutic availability includes Initial therapeutic availability resulting from release of non-encapsulated therapeutic contained within the embedded therapeutic region 502 and dispersed therapeutic region 504 following matrix hydration. Also, the sequential therapeutic availability includes Sustained therapeutic availability resulting from delayed degradation of biodegradable carriers forming the encapsulated therapeutic region 506. The sequential release behavior supports maintenance of therapeutic exposure within intradermal tissue across a defined treatment window while avoiding administration of excessive peak doses associated with injection-based delivery.

The insertion of multiple microneedles within the plurality of microneedles 104 distributes therapeutic deposition across an administration site. Dissolution occurring after successful insertion promotes localized intradermal placement of therapeutic material corresponding to positions of individual microneedles. The uniform dissolution behavior across the microneedle array reduces variability associated with heterogeneous insertion performance. Distributed intradermal deposition improves dose consistency relative to delivery systems relying upon single injection sites or retained depot structures. Complete dissolution of the plurality of microneedles 104 enables removal of external portions of the dissolvable microneedle patch 100 after administration without leaving persistent structural components within skin. Absence of retained hardware supports repeat administration over multiple treatment intervals. The dissolution and release behavior of the dissolvable microneedle patch enables delivery of large molecule therapeutics using a minimally invasive wearable device while maintaining compatibility with protocol-driven hormone administration schedules.

Accordingly, FIG. 5 illustrates spatial distribution of therapeutic material within each microneedle and functional interaction between matrix dissolution and biodegradable carrier degradation following intradermal insertion. The illustrated configuration enables administration of large molecule therapeutics in native molecular form and providing controlled therapeutic exposure through a fully dissolvable microneedle structure.

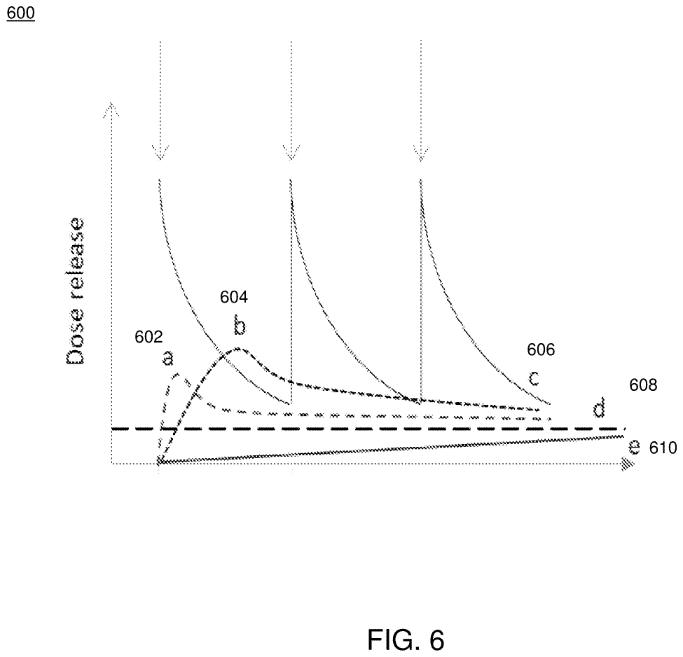

FIG. 6 illustrates a schematic representation 600 of dissolution and therapeutic release of the plurality of microneedles 104 following insertion into skin layers, in accordance with various embodiments of the present disclosure. The dissolution behavior of the plurality of microneedles 104 and corresponding therapeutic release following insertion into skin layers is schematically represented as a relationship between administered dose and release over time. The illustrated profiles represent therapeutic release resulting from dissolution of the microneedle matrix and release of encapsulated therapeutic material after intradermal placement.

Following application of the dissolvable microneedle patch 100 onto a skin surface, the plurality of microneedles 104 penetrate the stratum corneum and extend into epidermal or dermal tissue regions. Upon insertion, interstitial fluid present within skin contacts external surfaces of the plurality of microneedles 104 and initiates hydration of matrix materials forming the microneedles.

Hydration of the non-crosslinked hyaluronic acid and methylcellulose matrix progressively reduces structural rigidity of the plurality of microneedles 104. Dissolution proceeds from outer microneedle surfaces toward interior regions of the microneedle body. Dissolution behavior governing therapeutic release is adjustable through modification of polymer composition and microneedle dimensional parameters. Dissolution of the matrix results in release of therapeutic material incorporated within the embedded therapeutic region 502 and dispersed therapeutic region 504 described with reference to FIG. 5.

A first release profile 602 represents release of non-encapsulated therapeutic material following hydration and dissolution of the microneedle matrix. Therapeutic material located within embedded and dispersed regions becomes exposed to interstitial fluid as dissolution progresses, resulting in relatively rapid availability of therapeutic following insertion.

Release associated with the first release profile 602 corresponds to administration occurring shortly after insertion of the plurality of microneedles 104 into skin layers. The release behavior enables establishment of therapeutic levels without reliance on injection-based bolus delivery.

A second release profile 604 represents continued therapeutic release associated with progressive dissolution of remaining portions of the microneedle matrix. As dissolution advances through interior regions of the plurality of microneedles 104, additional therapeutic material incorporated within the matrix becomes available for intradermal administration.

The second release profile 604 reflects sustained release occurring during ongoing dissolution of the microneedle structure. The dissolution rate depends upon microneedle dimensions, matrix composition, hydration rate, and physiological conditions present within skin tissue.

A third release profile 606 represents delayed therapeutic release associated with degradation of biodegradable polymer material forming encapsulated therapeutic regions 306. Following dissolution of the microneedle matrix, biodegradable polymer structures containing encapsulated therapeutic material become exposed within intradermal tissue.

Degradation of biodegradable polymer material under physiological conditions releases encapsulated therapeutic over an extended duration following insertion. Release associated with the third release profile 606 occurs independently of structural dissolution of the plurality of microneedles 104.

In certain embodiments, therapeutic release from encapsulated material occurs over a period ranging between approximately 1 day and 14 days.

FIG. 6 further illustrates combined therapeutic exposure resulting from interaction of multiple release contributions. A cumulative therapeutic profile 608 represents overall therapeutic availability resulting from dissolution-driven release together with release from encapsulated therapeutic material.

A reference profile 610 represents therapeutic exposure associated with conventional administration approaches requiring repeated injections to maintain therapeutic levels. The cumulative therapeutic profile 608 demonstrates maintenance of therapeutic exposure over time without repeated administration events.

The illustrated release behavior results from coexistence of non-encapsulated therapeutic material and encapsulated therapeutic material within the plurality of microneedles 104, as previously described with reference to FIG. 5. Less than 100% of the therapeutic is encapsulated, thereby enabling dissolution-mediated release followed by extended release governed by biodegradable polymer degradation.

As dissolution progresses, the plurality of microneedles 104 fully dissolve within skin tissue, leaving no rigid or non-dissolving microneedle structures embedded within the subject. External portions of the dissolvable microneedle patch 100, including the base layer 102, remain removable following completion of administration.

Complete dissolution eliminates generation of sharps waste and enables repeat administration without accumulation of retained delivery hardware within tissue.

The dissolution and therapeutic release behavior illustrated in FIG. 6 enables administration of large molecule therapeutics through staged release resulting from dissolution of microneedle matrix material and degradation of biodegradable polymer associated with encapsulated therapeutic material.

The combined release behavior supports maintenance of therapeutic exposure within intradermal tissue over a defined treatment interval while avoiding peak-dose variability associated with injection-based administration.

In an embodiment, following dissolution of the plurality of microneedles 104 and release of at least one large molecule therapeutic into intradermal tissue, the therapeutic becomes distributed within interstitial fluid present between cellular structures of the skin.

Interstitial fluid functions as a transport medium facilitating movement of dissolved therapeutic molecules away from microneedle insertion sites. The released large molecule therapeutic diffuses within the interstitial fluid and undergoes uptake into local microvascular and lymphatic networks present within dermal tissue regions.

In certain embodiments, transport of the large molecule therapeutic occurs through capillary uptake and lymphatic absorption pathways naturally present within intradermal tissue. The dermis includes dense vascularization and lymphatic vessels capable of absorbing macromolecular substances that are not capable of passive diffusion across intact skin.

Following uptake into lymphatic or capillary circulation, the large molecule therapeutic enters systemic circulation and becomes available for interaction with target receptors or biological pathways associated with a therapeutic indication. Systemic distribution enables the therapeutic agent to produce a physiological response consistent with administration of the therapeutic through conventional injection-based delivery routes.

In certain embodiments, intradermal placement achieved using the plurality of microneedles 104 enables gradual absorption of the large molecule therapeutic from interstitial fluid, thereby reducing rapid concentration spikes associated with bolus injection while supporting maintenance of therapeutically effective exposure levels over time.

Absorption occurring through dermal microcirculation enables administration of biologically active large molecule therapeutics without chemical modification of the therapeutic agent and without reliance on deep tissue injection. The uptake pathway therefore supports generation of a therapeutic effect following minimally invasive intradermal administration using the dissolvable microneedle patch 100.

Accordingly, the release characteristics illustrated in FIG. 6 demonstrate coordinated burst release and sustained release of at least one large molecule therapeutic following intradermal administration. Dissolution of the microneedle matrix material within interstitial tissue enables controlled liberation of therapeutically active agents and avoids retention of solid delivery structures within the skin. The release profiles represented in FIG. 6 illustrate maintenance of therapeutic exposure levels relative to required therapeutic thresholds over an extended administration period. The dissolution and release mechanism therefore enables administration of large molecule therapeutics in a minimally invasive manner while supporting sustained therapeutic availability without reliance on repeated injections or retained implantable delivery systems, in accordance with various embodiments of the present disclosure.

### Experimental Data and Technical Evaluation

The following examples describe experimental evaluations conducted in relation to the dissolvable microneedle patch 100. The examples illustrate preparation, fabrication, material behavior, mechanical performance, drug incorporation strategies, release characteristics, and biological evaluation. The examples provide technical support for material selection, geometry selection, release behavior, and fabrication feasibility. The examples do not limit the scope of dissolvable microneedle patch 100 or the method of manufacturing described in FIG. 2.

### EXAMPLE 1—Matrix Composition Evaluation

Formulations containing hyaluronic acid and methylcellulose were prepared as candidate matrix materials for the plurality of microneedles 104. The high molecular weight hyaluronic acid, the low molecular weight hyaluronic acid, and the methylcellulose were combined at defined weight percentages. The polymer compositions were selected within ranges described in the specification. Multiple gel compositions were prepared and recorded. Evaluation determined that polymer composition directly influences gel behavior, viscosity, structural integrity, and castability. The results establish operable composition ranges suitable for fabrication of the plurality of microneedles 104. The matrix composition includes alternative polymer ratios within disclosed ranges.

### EXAMPLE 2—Preparation of Injectable Gels

Injectable gel formulations were prepared using hyaluronic acid, methylcellulose, or combinations thereof. Dry polymer powders were dissolved in heated water. The heating temperature was selected within ranges described for preparation step 202. The cooling to room temperature increased the viscosity and formed the hydrated matrix material. Mixed gel formulations were prepared by combining the dry powders prior to the hydration. Preparation methods yielded injectable gels capable of casting into the microneedle mold described in FIG. 2. The results demonstrate multiple preparation approaches suitable for forming the hydrated matrix material used to fabricate the dissolvable microneedle patch 100.

### EXAMPLE 3—Preparation of Microneedle Patches

Injectable gel formulations were transferred into a microneedle mold to form the plurality of microneedles 104 and the base layer 102. Casting was conducted through solution casting. Drying was conducted using air drying or freeze-drying. In some preparations, centrifugation was applied prior to drying to remove entrapped air. The term “solution casting” refers to deposition of hydrated polymer material into micro-scale cavities. Fabrication resulted in structured microneedle arrays extending from the base layer 102. The results demonstrate feasibility of forming the dissolvable microneedle patch 100 from the disclosed gel compositions using multiple fabrication techniques.

### EXAMPLE 4—Preparation of Drug-Loaded Injectable Gels

Active pharmaceutical ingredients were incorporated into injectable gel matrices. Incorporation occurred through direct mixing into the hydrated matrix material. Incorporation also occurred through encapsulation prior to mixing. Incorporation further occurred through a combination of direct and encapsulated loading. Multiple incorporation strategies were implemented without altering matrix structural integrity. The results demonstrate flexibility in drug loading approach for the dissolvable microneedle patch 100.

### EXAMPLE 5—Preparation of Drug-Loaded Microneedle Patches

Drug-loaded injectable gels were transferred into the microneedle mold. Drying was conducted through air drying or freeze-drying. Centrifugation was optionally applied before drying. Fabrication yielded the plurality of microneedles 104 containing incorporated active pharmaceutical ingredients. The results demonstrate that active pharmaceutical ingredients integrate into the dissolvable microneedle structures without preventing formation of mechanically stable microneedles.

### EXAMPLE 6—Influence of Composition on Gel Properties

Polymer composition was varied to evaluate gel injectability, gel-sol transition time, hardness, and firmness. Injectability assessment used syringe extrusion scoring. Hardness and firmness were assessed through deformation-based measurements. Evaluation determined that hyaluronic acid alone did not provide optimal gel properties. Also, the evaluation determined that methylcellulose improved gel behavior depending on viscosity and concentration. In addition, the evaluation determined that inclusion of the low molecular weight hyaluronic acid improved flow and injectability despite increased total solids content. The results demonstrate that composition influences mechanical and rheological properties of the hydrated matrix material.

### EXAMPLE 7—Influence of High Molecular Weight Hyaluronic Acid on Microneedle Integrity

Formulations containing varying amounts of high molecular weight hyaluronic acid were evaluated. Microneedle integrity was assessed based on spreadability, castability, flexibility, and strength. Evaluation was conducted at a fixed microneedle aspect ratio. The results demonstrate that high molecular weight hyaluronic acid content influences microneedle mechanical performance during fabrication.

### EXAMPLE 8—Influence of Needle Shape and Dimensions on Mechanical Integrity

Microneedles having different shapes were fabricated from a selected matrix formulation. Cone-shaped microneedles and square-shaped microneedles were evaluated. Mechanical performance assessment included castability, penetrability, piercibility, flexibility, and strength. The results demonstrate that square-shaped microneedles provided improved mechanical performance during penetration relative to conical microneedles. The results confirm that microneedle geometry selection influences mechanical stability of the plurality of microneedles 104.

### EXAMPLE 9—Sustained Release Behavior of Drug-Loaded Microneedle Patches

Drug-loaded dissolvable microneedle patch samples were incubated at 37° C. in water, phosphate-buffered saline, or saline. Drug release was quantified using UV-visible spectroscopy. Initial burst release occurred within approximately 1-30 minutes. Sustained release extended from approximately 30 minutes up to 14 days. Release behavior depended on drug loading concentration and encapsulation chemistry. The results demonstrate multi-phase release corresponding to matrix dissolution and biodegradable polymer degradation.

### EXAMPLE 10—Safety and Efficacy Evaluation

In vitro evaluation was conducted using ovarian and endometrial cell lines. Cells were incubated with prototype dissolvable microneedle patch 100 formulations. Cell viability and hormone production assays were conducted over time. Evaluation demonstrated maintenance of cell viability. Further, the evaluation demonstrated functional hormone response consistent with therapeutic intent. The results support safety and efficacy of disclosed formulations under in vitro conditions.

The examples demonstrate material feasibility, mechanical performance, fabrication capability, drug incorporation flexibility, sustained release behavior, and biological compatibility of the dissolvable microneedle patch 100. The examples provide technical support for ranges and mechanisms described in FIG. 1, FIG. 2, and FIG. 3. In yet another embodiment of the present disclosure, additional formulations, geometries, release profiles, and biological evaluations may be conducted within disclosed material and dimensional ranges.

The structure, the material composition, the dissolution behavior, and the release profile of the dissolvable microneedle patch 100 enable application beyond fertility-specific regimens. The following therapeutic applications illustrate non-limiting use contexts consistent with the disclosed structural and functional framework.

### Use Case 1—Luteal Phase and Menstrual Cycle Hormone Support

The dissolvable microneedle patch 100 is used for luteal phase hormone support. The luteal phase support includes administration of peptide or protein hormones during the post-ovulatory phase of the menstrual cycle. The luteal phase therapy requires repeat administration within defined dosing windows. Further, the luteal phase therapy requires consistent hormone exposure across a multi-day interval. The dissolvable microneedle patch 100 provides repeat administration without retained needles or solid implants. In addition, the dissolvable microneedle patch 100 is used for menstrual-cycle-related hormone therapies involving peptide or protein hormones. Menstrual-cycle-related therapies share dosing schedules and molecular characteristics similar to fertility hormone regimens. Further, dosing interval, hormone type, or exposure duration differs within disclosed structural and release ranges.

### Use Case 2—Perimenopausal and Menopausal Hormone Therapies

The dissolvable microneedle patch 100 is used for perimenopausal hormone therapy involving peptide or protein-based hormones. Also, the dissolvable microneedle patch 100 is used for menopausal hormone therapy involving peptide or protein-based hormones. Peptide or protein hormones used in menopausal management may exhibit limited oral bioavailability. Further, long-acting injectable depots may not align with preferred dosing paradigms.

### Use Case 3—Endocrine Disorders Requiring Peptide Hormone Replacement

The dissolvable microneedle patch 100 is used for endocrine disorders treated with peptide hormone replacement. The endocrine disorders include conditions involving pituitary or hypothalamic hormone supplementation. Peptide hormone replacement requires repeat administration within defined therapeutic windows. Further, the peptide hormones exhibit susceptibility to enzymatic degradation and limited passive dermal penetration. The dosing schedule and the release profile are adjusted to maintain hormone concentration within a defined therapeutic range.

### Use Case 4—Non-Human Reproductive and Breeding Applications

The dissolvable microneedle patch 100 is used for assisted reproductive applications in non-human subjects. The non-human subjects include veterinary species requiring peptide hormone delivery. The hormone delivery regulates ovulation, estrus, or reproductive cycle timing. Further, repeat administration occurs within defined breeding schedules. The dissolvable microneedle patch 100 is adapted for animal breeding programs involving hormone-controlled reproductive cycles. In addition, animal species, hormone type, and dosing paradigm vary within disclosed structural and release parameters.

The therapeutic use cases described above illustrate applications consistent with structural configuration of the base layer 102, the plurality of microneedles 104, and the solidified matrix. The dissolvable microneedle patch 100 supports the intradermal delivery of the peptide or the protein hormones across the defined dosing windows without requiring retained implants or chemically modified hormone analogs. Further, the dissolvable microneedle patch 100 is used in additional therapeutic contexts involving peptide or protein-based hormones within disclosed molecular weight, composition, and dissolution ranges.

1. A dissolvable microneedle patch for intradermal administration of a hormone to a subject, the microneedle patch comprising:
a substantially planar base layer; and
a plurality of microneedles extending from a surface of the base layer,
wherein each of the plurality of microneedles has a height of 1000-3000 μm100,
wherein each of the plurality of microneedles comprises a solidified matrix, wherein the matrix comprises:
(a) 0.5-20% w/w non-crosslinked high molecular weight hyaluronic acid having a molecular weight of 1000-1400 kDa;
(b) 0.5-20% w/w methylcellulose having a viscosity of 100-8000 cP; and
(d) 0-20% w/w non-crosslinked low molecular weight hyaluronic acid having a molecular weight of 20-1000 kDa,
wherein the matrix comprises the hormone and no added chemical crosslinking agents,
wherein less than 100% of the hormone is encapsulated in at least one biodegradable polymer dispersed within the matrix, wherein a non-encapsulated portion of the hormone is present within the matrix material, and an encapsulated portion of the hormone is contained within the biodegradable polymer, and
wherein the plurality of microneedles dissolve within 1-14 days following insertion into intradermal tissue and the biodegradable polymer degrades within 1-14 days following insertion.

2. The microneedle patch of claim 1, wherein the plurality of microneedles have a pitch in a range of 100-3000 μm and a base width of 100-500 μm.

3. The microneedle patch of claim 1, wherein the microneedle patch comprises 50-1000 microneedles.

4. The microneedle patch of claim 1, wherein the microneedles maintain structural integrity during insertion into intradermal tissue.

5. The microneedle patch of claim 1, wherein the base layer and the plurality of microneedles comprise the matrix and the plurality of microneedles are congruous extensions of the base layer.

6. The microneedle patch of claim 1, wherein the base layer comprises an adhesive surface configured to secure the microneedle patch to skin of the subject, wherein the adhesive surface forms a perimeter or partial perimeter around the base layer.

7. The microneedle patch of claim 1, wherein the microneedle patch further comprises a non-adhesive top layer.

8. The microneedle patch of claim 1, wherein each of the plurality of microneedles comprise a tapered tip having a contact area less than that of a 30-gauge hypodermic needle.

9. The microneedle patch of claim 1, wherein each of the plurality of microneedles comprise a tip portion that constitutes 5-95% of the total volume of the plurality of microneedles.

10. The microneedle patch of claim 1, wherein the plurality of microneedles are boreless.

11. The microneedle patch of claim 1, wherein the base layer has a surface area of approximately 4 cm×4 cm, 3 cm×3 cm, or 1.5 cm×1.5 cm.

12. The microneedle patch of claim 1, wherein the base layer further comprises at least one sensor configured to detect at least one of a hormone concentration in blood, interstitial fluid, or sweat, a biological marker associated with endocrine function, and a temperature of interstitial fluid.

13. The microneedle patch of claim 1, wherein the biodegradable polymer comprises at least one of polyethylene glycol (PEG), polyvinyl alcohol (PVA), poly(lactic-co-glycolic acid) (PLGA), polycaprolactone (PCL), poly(lactic acid) (PLA), polyanhydride, PMMA, PHEMA, polyurethanes, poly(amino acids), PVP, polysaccharides, proteins, or polynucleotides.

14. The microneedle patch of claim 1, wherein the hormone is a reproductive hormone selected from follicle stimulating hormone (FSH), luteinizing hormone (LH), gonadotropin releasing hormone (GnRH), progesterone, human chorionic gonadotropin (hCG), estrogen, or testosterone.

15. The microneedle patch of claim 1, wherein the microneedle patch is used for administration of 10-100 IU of the hormone.

16. The microneedle patch of claim 1, wherein the microneedle patch is used for administration of 40-60 IU of the hormone.

17. The microneedle patch of claim 1, wherein the microneedle patch is configured for replacement daily over 2-14 days.

18. The microneedle patch of claim 1, wherein the microneedle patch is for use with at least one intradermal or subdermal injection of at least one additional large molecule therapeutic.

19. A method of manufacturing a dissolvable microneedle patch for intradermal administration of a hormone to a subject, the method comprising:
a) preparing a matrix material by mixing non-crosslinked high molecular weight hyaluronic acid, methylcellulose, and at least one large molecule therapeutic with heated water to form a hydrated matrix material;
b) cooling the hydrated matrix material to room temperature; and
c) casting the cooled matrix material into a microneedle mold and drying the matrix material within the microneedle mold to form a plurality of microneedles extending from a base layer of the microneedle patch.

20. A method of delivering a hormone to a subject in need thereof, the method comprising:
applying a dissolvable microneedle patch to a skin of the subject, wherein the dissolvable microneedle patch comprises:
a substantially planar base layer; and
a plurality of microneedles extending from a surface of the base layer, wherein each of the plurality of microneedles has a height of 1000-3000 μm, wherein each of the plurality of microneedles comprises a solidified matrix, wherein the solidified matrix comprises:
(a) 0.5-20% w/w non-crosslinked high molecular weight hyaluronic acid having a molecular weight of 1000-1400 kDa;
(b) 0.5-20% w/w methylcellulose having a viscosity of 100-8000 cP; and
(c) 0-20% w/w non-crosslinked low molecular weight hyaluronic acid having a molecular weight of 20-1000 kDa, wherein the solidified matrix comprises the hormone and no added chemical crosslinking agents,
exerting sufficient force on the microneedle patch to cause the plurality of microneedles to penetrate the skin and enter intradermal tissue of the subject;
maintaining the microneedle patch in contact with the skin until at least a portion of the solidified matrix dissolves; and
removing the microneedle patch from the skin,
wherein less than 100% of the hormone is encapsulated in a biodegradable polymer, wherein the plurality of microneedles dissolve within 1-14 days following insertion and the biodegradable polymer degrades within 1-14 days following insertion.
